SEQUENCE LISTING (2 sequences)

SEQ ID NO: 1 - DNA, 11 bp, Artificial Sequence
  Hypothetical sequence for a sequence read, not corresponding to any known genetic source, constructed in silico, for purposes of explaining an algorithm
gaaaatcgatc

SEQ ID NO: 2 - DNA, 11 bp, Artificial Sequence
  Hypothetical sequence for a sequence read, not corresponding to any known genetic source, constructed in silico, for purposes of explaining an algorithm
gaaatcgattc

(19) United States
(12) Patent Application Publication
(10) Pub. No.: US 2020/0395098 A1
(43) Pub. Date: Dec. 17, 2020

(54) ALIGNMENT USING HOMOPOLYMER-COLLAPSED SEQUENCING READS

(71) Applicant: Pacific Biosciences of California, Inc., Menlo Park, CA (US)

(72) Inventor: Robert Grothe, Campbell, CA (US)

(21) Appl. No.: 16/794,696

(22) Filed: Feb. 19, 2020

Related U.S. Application Data
(60) Provisional application No. 62/812,191, filed on Feb. 28, 2019.

(51) Int. Cl. G16B 30/20; G16B 30/10

(52) U.S. Cl. CPC G16B 30/20; G16B 30/10

(57) ABSTRACT

The present disclosure provides, inter alia, methods, compositions, and computer implemented processes for resolving long and highly similar, but non-identical, genomic regions to improve assembly quality, especially for polyploid genomes. Aspects of the disclosure are draw to using exact string matching of homopolymer-collapsed sequence reads to determine whether two sequences overlap and thus represent the same genomic region (e.g., the same haplotype in polyploid genomes) or whether the sequences represent different genomic regions.

### CROSS REFERENCE TO RELATED APPLICATION

This application claims priority to U.S. Provisional Patent Application No. 62/812,191, filed Feb. 28, 2019, the disclosure of which is hereby incorporated by reference herein in its entirety for all purposes.

### REFERENCE TO SEQUENCE LISTING

The Sequence Listing text copy submitted herewith via EFS-Web was created on Sep. 1, 2020, is entitled 0671915120US ST25.txt, is 1 kilobyte in size and is herein incorporated by reference in its entirety.

### BACKGROUND

Genome sequence assembly refers to the determination of the nucleotide sequence of each of the genome's chromosomes by a process in which each chromosome is broken into smaller genomic fragments, the nucleotide sequence of each genomic fragment is “read” rendering the fragment sequence into a read sequence, and then the read sequences are assembled. Multiple copies of the genomic DNA are required for assembly. These multiple copies can be obtained either from multiple cells from the same organism, assumed to have identical genomic DNA, or by replication (e.g., PCR amplification) of the genome contained in a single cell. When the same genomic locus is covered by two distinct fragments, the two fragments are said to “overlap”. The nucleotide sequences of overlapping fragments also overlap, in the sense that they share a common subsequence. If the common subsequence shared by the overlapping fragments occurs uniquely in the genome, it is possible to detect the overlap between these fragments from reads of these fragments. In this case, if two reads also share a common nucleotide sequence that extends to one end of each read, then it is correctly inferred that the two reads were derived from a pair of overlapping genomic fragments. The two reads can be “overlapped” by superimposing the common sequence. A graph structure can be formed, in which the vertices (reads) are connected by edges between “overlapped” reads. Each edge represents the assertion that the two reads were derived from genome fragments that contain the same genomic locus. In a valid assembly, each connected component represents overlapping genome fragments derived from the same chromosome. A contig can be formed from each connected component by aligning the reads, superimposing positions in the reads that correspond to the same position in the genome. In the absence of read errors, the nucleotide identity at each position can be correctly determined. Given read errors, the “pileup” of many overlapping reads at each genomic position allows the draft assembly to be polished to high consensus accuracy using redundancy to suppress read errors.

While the assembly process is conceptually simple, correct overlap detection throughout an entire genome has proved difficult, especially for genomes containing long regions of repetitive sequence. Assembly is fundamentally limited by the accuracy of detecting overlapping genomic fragments from their reads. False-positive overlap errors occur when reads from two distinct loci are mistakenly identified as being derived from the same locus. False positives may arise when two distinct loci have long regions of identical or nearly identical sequence. False-negative overlap errors occur when reads from overlapping genomic fragments are mistakenly identified as being derived from distinct loci. False negatives may arise when read errors obscure the common nucleotide sequence shared by overlapping genomic fragments. Both types of overlap errors, if not subsequently corrected, lead to assembly errors. False-positive overlaps can cause fusion of chromosomes or, more frequently, expansion or collapse of repetitive elements. False-negative errors, especially systematic ones, may lead to breaks in the assembly, where a single chromosome is represented by multiple disjoint contigs, which can be accompanied by the loss of some loci at contig boundaries.

The present disclosure addresses, inter alia, the challenges posed by the presence of highly similar, but not identical, sequences in haploid and polyploid genomes to the assembly of the genomes.

### SUMMARY

The present disclosure provides, inter alia, methods, compositions, and computer implemented processes for resolving long and highly similar, but non-identical, genomic regions to improve assembly quality, especially for polyploid genomes. At a fundamental level, this includes determining whether two sequences overlap or not, i.e., whether the sequences represent the same genomic region—and in polyploid genomes, the same haplotype at that region—or whether the sequences represent different genomic regions—or different haplotypes.

Aspects of the present disclosure include a method for assembling a genome or a genomic region, the method comprising: obtaining a plurality of sequence reads for genomic fragments from a genome of interest; generating a homopolymer-collapsed sequence (HCS) for each of the plurality of sequence reads and a corresponding homopolymer encoded sequence (HES); generating suffix/prefix exact string matches of the HCS reads, wherein the length of the exact string match is at or above a minimum length; generating trimmed HCS reads by removing any nucleotides for each of the plurality of HCS reads that are not part of a suffix/prefix exact string match with another HCS read; generating a first directed overlap graph from the trimmed HCS reads; identifying the connected components in the second directed overlap graph; generating a multiple sequence alignment for each of the connected components, wherein the positions in each trimmed HCS read are labeled with consecutive integer values so that aligned positions in any two trimmed HCS reads are assigned the same integer value; pruning merged nodes from the second directed overlap graph based on the multiple sequence alignment; generating a homopolymer-collapsed consensus sequence by concatenating the basecall at each aligned position in the multiple sequence alignment of the trimmed HCS reads; associating a vector of homopolymer lengths for each position in the homopolymer-collapsed consensus sequence, wherein: (i) the number of elements in the vector is the number of trimmed HCS reads covering that position in the multiple sequence alignment, and (ii) each component of the vector is the length of the homopolymer in the corresponding HES at that position; assigning a consensus homopolymer length for each position in the homopolymer-collapsed consensus sequence as the floor of the median of the components of the vector of homopolymer lengths associated with that position; and replacing each position in the homopolymer-collapsed consensus sequence with a homopolymer string formed by N successive copies of nucleotide at that position, wherein N is the assigned consensus homopolymer length calculated for that position, to generate a homopolymer-expanded consensus sequence, thereby assembling the genome or genomic region of the genome of interest.

In certain embodiments, prior to generating HCS reads, the method further comprises generating reverse complement sequences of each of the plurality of sequence reads.

In certain embodiments, the overlap region has a minimum length is from 0.5 kb to 10 kb. In certain embodiments, the overlap region has a minimum length is from 5 kb to 8 kb. In certain embodiments, the overlap region has a minimum length is from 6 kb to 7 kb. In certain embodiments, the minimum length that is at least half the length of the average length of the HCS reads.

In certain embodiments, the plurality of sequence reads are generated in a single molecule sequencing-by-synthesis reaction. In certain embodiments, the single molecule sequencing by synthesis reaction is a Single Molecule, Real-Time (SMRT®) Sequencing reaction. In certain embodiments, the plurality of sequence reads are generated in a single molecule nanopore sequencing reaction.

In certain embodiments, the plurality of sequence reads is a plurality of single molecule consensus sequences (SMCSs). In certain embodiments, the SMCSs are generated from at least 8 subreads. In certain embodiments, the subreads are generated in a single molecule sequencing reaction from a concatemeric polynucleotide substrate. In certain embodiments, the subreads are generated in a single molecule sequencing-by-synthesis reaction. In certain embodiments, the subreads are generated in a single molecule nanopore-based sequencing reaction. In certain embodiments, the subreads are generated in a single molecule sequencing-by-synthesis reaction from a circular or topologically circular polynucleotide substrate.

In certain embodiments, the genome of interest is a human genome.

In certain embodiments, when the genomic sample comprises multiple different genomes, the method further comprising generating assemblies for multiple of the different genomes. In certain embodiments, the sample is a metagenomic sample comprising multiple microbial genomes.

In certain embodiments, HCSs that are not placed into a connected component are placed into a holding bin that is used to verify variant calls in the assembly.

In certain embodiments, the plurality of sequence reads are pre-selected to map to one or more genomic regions of interest prior to generating the HCSs. In certain embodiments, the pre-selection mapping is done with a low-stringency sequence similarity search. In certain embodiments, the one or more genomic regions of interest comprises first and second genomic loci having high sequence similarity to one another. In certain embodiments, the separate consensus sequences are generated for the first and second genomic loci. In certain embodiments, the one or more genomic regions of interest comprises a genomic locus having a highly repetitive region.

In certain embodiments, the method is a method for de novo genome assembly. In certain embodiments, the de novo genome assembly is a fully or partially haplotype resolved assembly of a polyploid genome.

Aspects of the present disclosure include a system for determining a consensus sequence, comprising: a memory; input/output; and a processor coupled to the memory, wherein the system is configured to: receive a plurality of sequence reads for genomic fragments from a genome of interest; generate a homopolymer-collapsed sequence (HCS) for each of the plurality of sequence reads and a corresponding homopolymer encoded sequence (HES); generate suffix/prefix exact string matches of the HCS reads, wherein the length of the exact string match is at or above a minimum length; generate trimmed HCS reads by removing any nucleotides for each of the plurality of HCS reads that are not part of a suffix/prefix exact string match with another HCS read; generate a first directed overlap graph from the trimmed HCS reads; identify the connected components in the second directed overlap graph; generate a multiple sequence alignment for each of the connected components, wherein the positions in each trimmed HCS read are labeled with consecutive integer values so that aligned positions in any two trimmed HCS reads are assigned the same integer value; prune merged nodes from the second directed overlap graph based on the multiple sequence alignment; generate a homopolymer-collapsed consensus sequence by concatenating the basecall at each aligned position in the multiple sequence alignment of the trimmed HCS reads; associate a vector of homopolymer lengths for each position in the homopolymer-collapsed consensus sequence, wherein: (i) the number of elements in the vector is the number of trimmed HCS reads covering that position in the multiple sequence alignment, and (ii) each component of the vector is the length of the homopolymer in the corresponding HES at that position; assign a consensus homopolymer length for each position in the homopolymer-collapsed consensus sequence as the floor of the median of the components of the vector of homopolymer lengths associated with that position; and replace each position in the homopolymer-collapsed consensus sequence with a homopolymer string formed by N successive copies of nucleotide at that position, wherein N is the assigned consensus homopolymer length calculated for that position, to generate a homopolymer-expanded consensus sequence; and provide the homopolymer-expanded consensus sequence to a user, thereby assembling the genome or genomic region of the genome of interest.

In certain embodiments, the system is further configured to perform the method according to any one of the embodiments above and output the results of the method to a user.

### DESCRIPTION OF THE FIGURES

FIG. 1 shows a schematic of the process of generating a SMCS read from a SMRTBELL® polynucleotide substrate (a double-stranded polynucleotide with hairpin adapters at both ends).

FIG. 2 shows an example of two overlapping genomic fragments and two reads derived from these genomic fragments that share a common subsequence.

FIG. 3 shows an example of two genomic fragments from distinct loci that share a common subsequence and an alignment of two reads derived from these fragments.

FIG. 4 shows two reads derived from a genomic fragment that contains a tandem repeat and two alignments of these reads.

FIG. 5 shows a diploid genome, two genomic fragments from the maternal copy of chromosome 2, and an alignment of two reads derived from these fragments.

FIG. 6 shows two genomic fragments derived the paternal and maternal copies of chromosome 2 and an alignment of two reads derived from these fragments.

FIG. 7 shows two overlapping genomic fragments and two pairs of reads derived from these fragments. The first pair is error-free, but the second read in the second pair contains a homopolymer deletion.

FIG. 8 is an illustration of the approximate orthogonality between signal—the biological variation between two highly similar sequences, which is often single nucleotide variation—and noise, read errors that confound the identification of overlapping genomic fragments, which is often homopolymer indels.

FIG. 9 shows two overlapping genomic fragments, two reads derived from these fragments, the second of which contains a homopolymer deletion, and an alignment of homopolymer-collapsed sequences derived from the reads.

FIG. 10 shows an example of how a read corrupted by a homopolymer can be “perfected” by homopolymer collapse. The homopolymer-collapsed sequence of the read matches the homopolymer-collapsed sequence of the genomic fragment from which the read is derived, masking out the indel error in the read.

FIG. 11 shows an example of filtering out homopolymer indel errors to identify a pair of overlapping reads and to avoid a false overlap with a read from a highly similar genomic fragment from a distinct allele.

FIG. 12 shows a diagram of exact string matching and the multiple sequence alignment between “perfected” reads.

FIG. 13, FIG. 14, and FIG. 15 show an algorithmic workflow for using HCSs to separate SMCSs into haplotypes, calling consensus for the haplotypes, calling consensus lengths for homopolymer regions in the consensus sequences to generate a homopolymer-expanded consensus sequence, and calling homozygous and heterozygous variants by comparing to a reference genome, where in some cases, previously excluded HCSs can be used for variant call validation.

FIG. 16 shows how a homozygous region can induce the undesired merging of two distinct haplotypes into a single connected component, that the haplotypes can be separated, but in the process, the haplotypes are fractured into smaller haplotigs, whose connectivity cannot be resolved without an SMCS read that fully spans the homozygous region. The process of removing merged nodes (i.e., node C) is sometimes referred to as “pruning” herein.

FIG. 17 shows how SMCS reads that span a homozygous region can resolve haplotypes. This is also a pruning process. The process of removing merged nodes (i.e., node C) is sometimes referred to as “pruning” herein.

FIG. 18 shows histograms of the lengths of the SMCS reads, the lengths of HCSs derived from these reads, and the ratios of the length of each HCS to the SMCS read from which it was derived.

FIG. 19 shows the multiple sequence alignment of 11 homopolymer-collapsed SMCS reads from a single haplotype of SMN2.

FIG. 20 shows the multiple sequence alignment of 51 homopolymer-collapsed SMCS reads in which two haplotypes of SMN1 are merged.

FIG. 21 shows the diploid assembly of 100 SMCS reads mapped to the SMN1 and SMN2 sequences in the human genome reference GrCh38.

### DETAILED DESCRIPTION

The present disclosure provides, inter alia, improved processes for resolving long and highly similar, but non-identical, genomic sequences to improve genome assembly quality, especially for polyploid genomes. In general, this process includes filtering out a predominant form of sequencing error that confounds genome assembly and enforcing exact string matching of the filtered reads to prevent the overlapping of reads derived from highly similar genomic fragments from different loci or different haplotypes.

### Definitions

The term “genomic fragment” is used herein to refer to a single-stranded or double-stranded DNA molecule that was extracted from a cell and broken off from the chromosome in which it resided, or alternatively, copies of such a molecule formed by replication (e.g., PCR or linear amplification). A genomic fragment is identified by a genomic locus—its original position in a chromosome, its nucleotide sequence, and, in polyploid genomes, a haplotype. Two genomic fragments are “overlapping” when the two fragments share a common genomic locus and, in polyploid genomes, belong to the same haplotype. The nucleotide sequences of overlapping genomic fragments are also overlapping; that is, the two nucleotide sequences share a common subsequence, corresponding to the genomic locus that is shared by the overlapping genomic fragments. However, the converse is not true. Two genomic fragments whose sequences share a common subsequence are not necessarily “overlapping” because it is possible that the common subsequence occurs at two distinct genomic loci or, in polyploid genomes, at the same locus but in different haplotypes. A genomic fragment can be derived from any source desired by a user (e.g., any animal, plant, fungus, single-celled organism, etc.). In some cases, a library of polynucleotide substrates may be derived from multiple different organisms, e.g., multiple different human samples or a metagenomic sample containing a mixture of different organisms. The genomic fragment can be the product of an amplification process (e.g., by PCR or linear amplification), native/non-amplified polynucleotides, or a combination of both (e.g., a polynucleotide substrate with an amplified genomic fragment and a non-amplified genomic fragment or a double stranded region of interest with a native strand and a complementary strand that was produced by amplification). No limitation in this regard is intended.

The term “polynucleotide substrate” is used herein to refer to a polynucleotide that includes a genomic fragment (or copy thereof) in a form that can be sequenced by a sequencing platform, regardless of the sequencing platform used. In certain embodiments, polynucleotide substrates include functional domains in addition to a genomic fragment (e.g., synthetic or otherwise engineered sequences and/or functional moieties) that aid in obtaining and/or analyzing the sequence of the genomic fragment. Examples of such functional domains include, but are not limited to, one or more of: primer binding sites, binding sites for motor proteins (e.g., as employed in certain nanopore sequencing technologies), capture primer binding sites, capture moieties (e.g., cholesterol, biotin, avidin/streptavidin, etc.), sequencing primer binding sites, barcodes, registration sequences, unique molecular identifiers, detectable labels, or any other convenient sequences or moieties. Such additional sequences and moieties can be provided by attaching adapters to genomic fragments, e.g., via ligation, amplification, etc., as commonly done in the art. Libraries of polynucleotide substrates for genomic fragments of interest, e.g., entire genomes, are routinely generated and analyzed in the art.

The present disclosure uses the term “region of interest” to refer to a subset of an entire genome to which the disclosed method can also be applied. For example, a “region of interest” may include one or more genes either as a contiguous block or multiple blocks. No limitation in this regard is intended.

The present disclosure uses the term “single-molecule consensus sequence” (SMCS) to refer to a consensus sequence obtained by analyzing multiple sequence reads of a genomic fragment. Each complete sequence read for the genomic fragment, excluding any sequences of flanking adapter polynucleotides, is referred to herein as a “subread”. Because of differences in the configurations of polynucleotide substrates and/or the sequencing technologies employed, a set of subreads for a region of interest can include subreads for (i) only a single strand of a polynucleotide or (ii) two complementary strands of a polynucleotide. For example, a polynucleotide substrate for which sequence data is desired might include multiple linear head-to-tail copies of a genomic fragment that, when sequenced, provides a set of subreads, one for each copy, representing the same original genomic fragment (e.g., a concatemeric polynucleotide substrate generated by rolling circle amplification of a circular polynucleotide containing a genomic fragment). Conversely, when a double-stranded genomic fragment with hairpin adapters at both ends is sequenced using a long-read sequencing-by-synthesis method (e.g., SMRTBELL® polynucleotide substrates used in SMRT® Sequencing that are structurally linear but topologically circular), a set of subreads is produced that includes subreads for the forward strand of the double-stranded genomic fragment and its complementary reverse strand. Both forward and reverse strand subreads can be analyzed to generate a consensus sequence for the genomic fragment. It is noted that the underlying sequencing methodology does not necessarily determine whether subreads for only a single strand or for complementary strands are obtained. For example, rolling circle amplification of a SMRTBELL® polynucleotide can produce a linear polynucleotide substrate that when sequenced using nanopore sequencing technology will return subreads of the two complementary strands. In addition, a structurally circular double-stranded polynucleotide substrate containing a genomic fragment (similar in topology to a bacterial plasmid) that is sequenced using a sequencing-by-synthesis method will return subreads of only one strand of the genomic fragment.

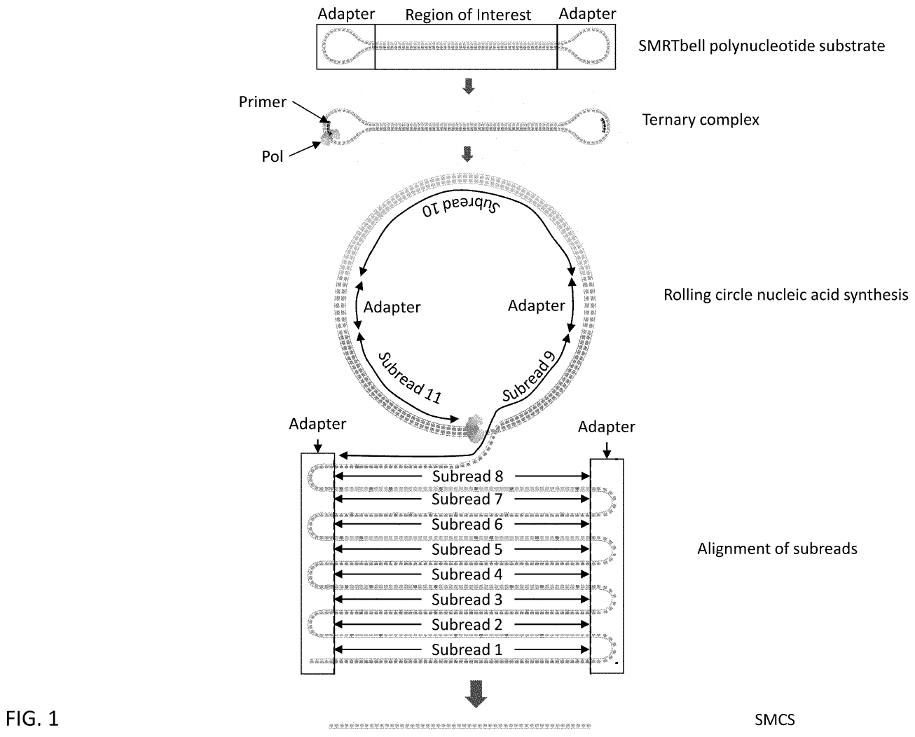

FIG. 1 provides a schematic for how SMCS reads are generated from a SMRTBELL® polynucleotide substrate in a SMRT® Sequencing reaction. On the top of FIG. 1, a SMRTBELL® polynucleotide substrate having a double-stranded DNA genomic fragment and two terminal hairpin adapters is shown. While only one polynucleotide substrate is shown, it should be clear that a SMRTBELL® library contains a population of SMRTBELL® polynucleotide substrates having the same general structure with various different, and generally overlapping, genomic fragments. This polynucleotide substrate is combined with a sequencing primer and polymerase under conditions to form a ternary complex that is competent for nucleic acid synthesis. The ternary complex is sequenced in a sequencing-by-synthesis SMRT® Sequencing reaction (Pacific Biosciences of California, Inc.), where the addition of each base is recorded in a single long sequencing read. Because the polynucleotide substrate is topologically circular, once the polymerase has traversed the entire polynucleotide substrate for the first time, it enters rolling circle amplification (RCA). The entire length of the single long sequencing read is called a “polymerase read” and includes all sequence data derived from the multiple passes of both the genomic fragment and the adapters. Each subread for both strands of the genomic fragment in the polymerase read is identified by removing adapter sequences. Each subread in FIG. 1 is labeled in the order it was generated. (Note that subread 11 is still being generated). Given the topology of the SMRTBELL® polynucleotide substrate, the odd subreads (i.e., subreads 1, 3, 5, 7, 9, and 11) represent sequence derived from one strand of the double-stranded genomic fragment in the polynucleotide substrate while the even subreads (i.e., subreads 2, 4, 6, 8, and 10) represent sequence derived from the other, complementary strand of the double-stranded genomic fragment in the polynucleotide substrate. Subreads 1 through 8 are aligned in FIG. 1 to emphasize this point (with the beginnings of subread 9 being aligned as the synthesized strand is being displaced from the polynucleotide substrate by the polymerase). After acquisition of the data for the subreads, a SMCS read for the genomic fragment in the polynucleotide substrate is generated. The quality value (QV) of a SMCS read depends on the accuracy of the polymerase read and the number of subreads used to generate the SMCS. Currently, an SMCS generated from 10 subreads on the SEQUEL® Sequencing platform achieves QV30 (see FIG. 1b in Wenger, A., et al., Jan. 13, 2019 “Highly-accurate long-read sequencing improves variant detection and assembly of a human genome” BioRxiv, doi.org/10.1101/519025; hereby incorporated herein by reference in its entirety for all purposes).

As indicated above, any method for generating SMCSs for a genomic fragment using a single-molecule sequencing platform may be used in the assembly method disclosed herein. As such, the term SMCS can be applied to data obtained using any single-molecule sequencing platform, e.g., the sequencing of SMRTBELL® polynucleotide substrates in Single Molecule, Real-Time (SMRT®) Sequencing from Pacific Biosciences, genomic fragments used in nanopore sequencing platforms, e.g., from Oxford Nanopore Technologies, Genia, and the like, or any other convenient single molecule sequencing platform. For example, SMCS reads can be generated using subreads from nanopore-based single-molecule sequencing data for concatemers formed from multiple copies of genomic fragments (e.g., as described in Volden et al., PNAS 2018, v115 (39), p. 9726-9731 “Improving nanopore read accuracy with the R2C2 method enables the sequencing of highly multiplexed full-length single-cell cDNA”, incorporated herein by reference in its entirety) or polynucleotide substrates having unique molecule identifiers (UMIs). No limitation in this regard is intended. Thus, any consensus sequence generated from single-molecule sequencing data for multiple subreads for a single genomic fragment, or copy/copies thereof, is encompassed in this term. With respect to SMRT® Sequencing, an SMCS represents the consensus sequence determined using subreads taken from a single SMRTBELL® polynucleotide substrate sequenced in a single zero-mode waveguide (ZMW) in a sequencing chip (as described above for FIG. 1). With respect to nanopore sequencing platforms, an SMCS represents the consensus sequence determined using subreads from a single original genomic fragment sequenced in either a single nanopore, e.g., a single polynucleotide substrate containing linked complementary strands and/or repeats derived from the single original genomic fragment (a “concatemer” as described above), or from multiple nanopores, e.g., separate copies of the same original genomic fragment sequenced in multiple different nanopores, where for example each copy is tagged with a UMI. For examples of single molecule sequencing platforms and methods, see the following U.S. patents and U.S. patent application Publications, each of which is incorporated herein by reference: U.S. Pat. No. 8,324,914, US2013/0244340, US2015/0119259, US2010/0196203, US2011/0229877, US2016/0162634, U.S. Pat. No. 7,315,019, US2009/0087850, and US2018/0023134.

The term “homopolymer-collapsed sequence” or “HCS” as used herein refers to a sequence that is derived from a parent sequence in which each instance of multiple consecutive identical nucleotides in the parent sequence is replaced by a single nucleotide of the same type. For example, the HCS of the polynucleotide sequence AATGGGCCG is ATGCG. Thus, “homopolymer collapse”, “collapsing homopolymers”, and the like are used to describe the process of creating a HCS from a parent sequence (a non-HCS).

A “homopolymer indel error” refers to a type of sequencing error in which a nucleotide that is identical to an adjacent, and correct, nucleotide in the read is inserted or deleted in the sequence read. For example, inserting an erroneous G into a sequence read next to a correct G, thereby resulting in a GG read when the correct read is a single G, is a homopolymer indel error. As another example, deleting a G from a four G stretch, thereby resulting in a GGG read instead of the correct GGGG read, is also a homopolymer indel error. Homopolymer indel errors may insert or delete more than a single nucleotide that is identical to an adjacent, and correct, nucleotide in the read, e.g., a homopolymer indel of 2, 3, or 4 nucleotides. As described herein, homopolymer indel errors in original sequence reads are filtered out by the process of forming corresponding HCSs (i.e., homopolymer collapse). Thus, homopolymer collapse transforms a sequencing read that contains a homopolymer indel error, i.e., one that is different from the genomic fragment from which it was derived, into a sequence (an HCS) that is identical to the HCS of the genomic fragment from which the sequence was derived.

A “perfected” sequence read is a sequence read whose homopolymer-collapsed sequence (HCS) is identical to the HCS of the genomic fragment from which it was derived. Indel errors in homopolymers in the sequence read are masked out by homopolymer collapse. If the only errors in a sequence read are homopolymer indels, then the read is perfected by homopolymer collapse.

Issues with Genome Assembly

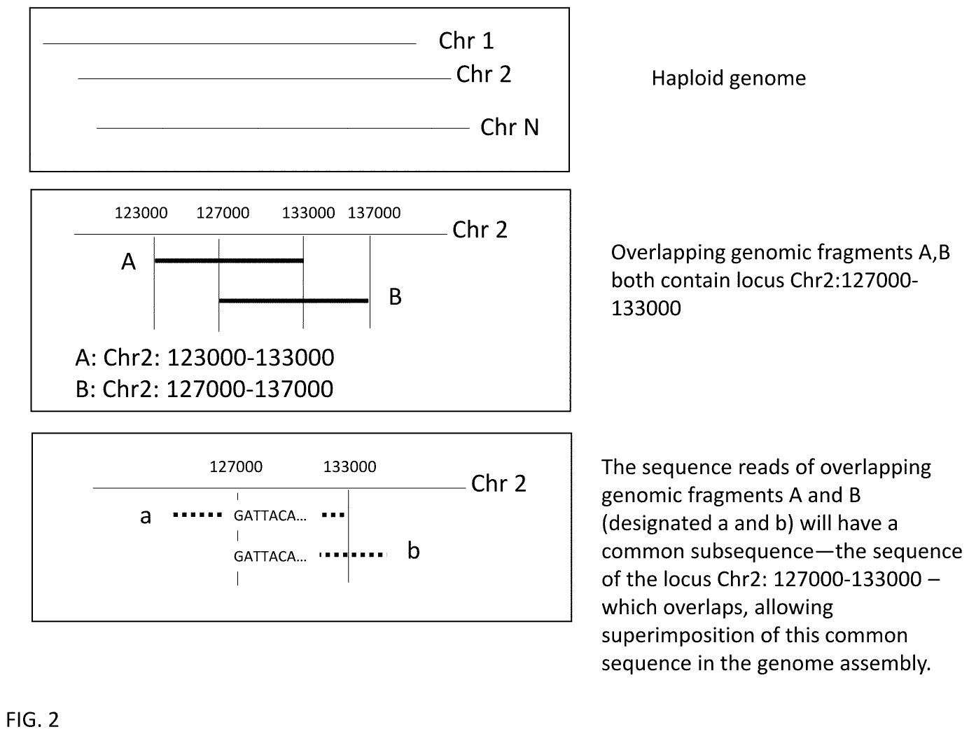

As noted above, genome assembly relies on the correct overlapping of sequence reads derived from distinct genomic fragments. When sequence reads from two independent genomic fragments share a common nucleotide sequence that extends to one end of each read (making a “dovetail” alignment), then it is correctly inferred that the two reads were derived from a pair of overlapping genomic fragments. The two sequencing reads can thus be overlapped by superimposing this common sequence. FIG. 2 provides a simple diagram showing how two genomic fragments (A and B in the second panel) from a chromosome of a haploid genome (indicated in the top panel) that include the same locus overlap. In this diagram, genomic fragment A includes nucleotides 123000 to 133000 from chromosome 2 (Chr2:123000-133000) while genomic fragment B includes nucleotides 127000 to 137000 from chromosome 2 (Chr2: 127000-137000). These genomic fragments both contain nucleotides 127000 to 1333000 (locus Chr2:127000-133000). Therefore, when these genomic fragments are sequenced (sequences a and b in the lower panel), their respective sequence reads will include a common overlapping subsequence, i.e., the sequence of Chr2: 127000-133000, which allows them to be superimposed in the genome assembly process.

When the common subsequence of two genomic fragments occurs only once in the genome, the overlap between the genomic fragments can be correctly inferred from the sequence reads of these fragments (as represented in FIG. 2). However, because genomes often comprise numerous repetitive elements, in which the same or highly similar sequence occurs at multiple distinct loci in the genome, the genome assembly process can be confounded. For example, many repetitive elements (even those that are not identical) share such high sequence similarity that their differences are not readily detected from their sequencing reads. In addition, long contiguous regions of repetitive sequence, e.g., long stretches of a 5-base sequence, can cause assembly errors. Therefore, the underlying assumption that sequence reads sharing a common sequence are necessarily derived from genomic fragments arising from the same locus can be rendered invalid by repetitive elements. As such, the detection of a region of identical (or nearly identical) sequence shared between a pair of reads is necessary for the two reads to represent overlapping genomic fragments, but it is not sufficient.

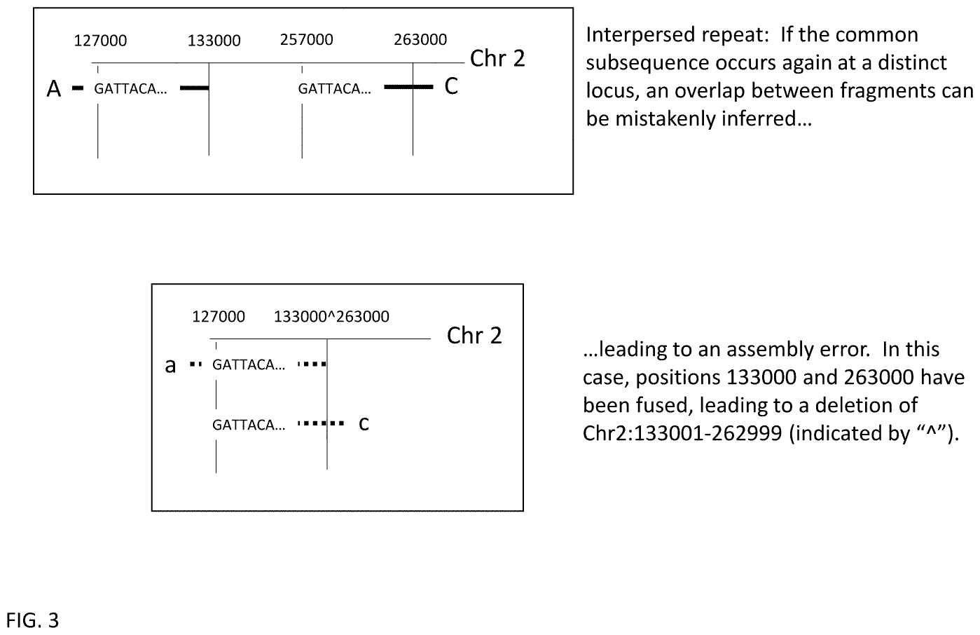

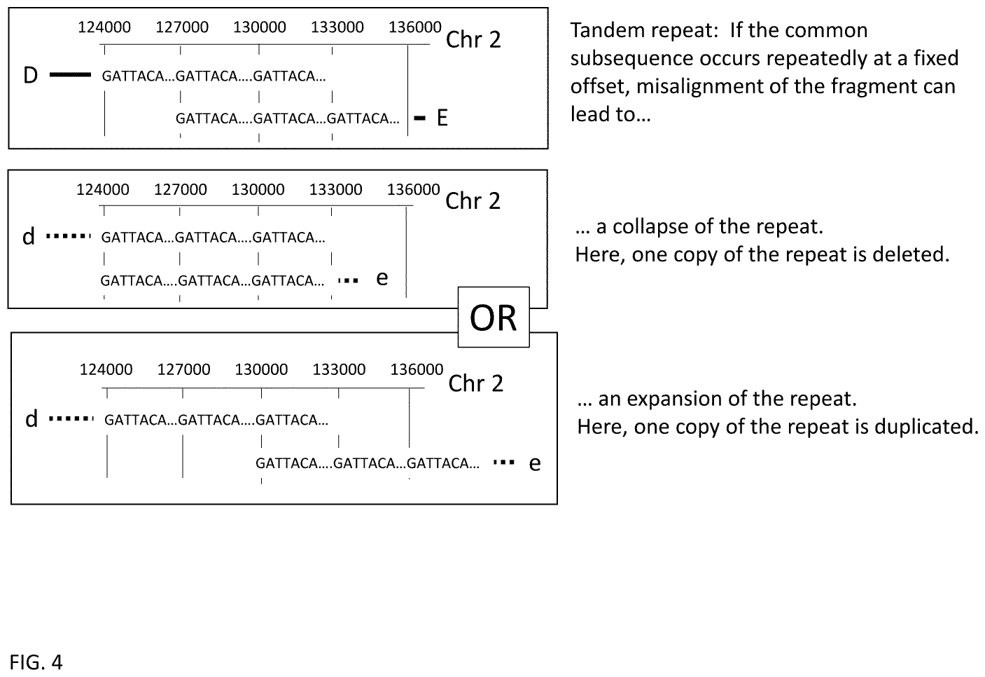

Tandem repeats and interspersed repeats are particularly troublesome regions that can cause errors or breaks in an assembly. A tandem repeat includes multiple consecutive copies of a repeating sequence motif while an interspersed repeat includes a sequence that occurs at two or more non-adjacent locations in the genome. FIG. 3 shows one example of how an interspersed repeat can negatively impact genome assembly. The top panel in FIG. 3 shows genomic fragments that include an identical subsequence of nucleotides but that are derived from different loci in the genome. Specifically, genomic fragment A ends with subsequence 127000-133000 (beginning somewhere upstream) and genomic fragment C begins with an identical subsequence from 257000-263000 (ending somewhere downstream). Sequence reads of these genomic fragments (a and c in the lower panel) can be overlapped in this identical subsequence region. However, this overlap leads to an incorrect inference regarding the underlying genome. Specifically, this overlap results in the deletion of nucleotides 1330001 to 262999 from the genome assembly. FIG. 4 shows one example of how tandem repeats can negatively impact genome assembly. In this figure, genomic fragments D and E include a common subsequence within a tandem repeat that, in total, has 4 copies of the same nucleotide sequence spanning nucleotides 124000-136000. Sequence reads of these genomic fragments (d and e in the lower panels) can be aligned such that one repeat is deleted, thus collapsing the repeat region (middle panel) or such that one repeat is added, thus expanding the repeat region (bottom panel).

The biological origin of tandem and interspersed repeats is most often one or more duplication events, followed by evolutionary divergence—the independent accumulation of mutations over subsequent generations. When two distinct loci share an identical sequence, correct assembly is possible only when there is at least one read that fully spans each occurrence of the common sequence. In this case, the sequences flanking the common sequence can be used to distinguish the loci. More commonly, genomes contain repetitive elements that originally arose from duplication or insertion of the same element multiple times in ancestral organisms, but those elements undergo mutations over many generations leading to sequence differences between them. In the absence of sequencing errors when genomic fragments are read, differences between genomic fragments would be detected, allowing distinct loci to be distinguished.

In interspersed repeats, the region flanking one repeat has low similarity to the respective region flanking a second repeat. It is thus possible to construct a contiguous assembly that bridges an interspersed repeat with two reads that overlap within the repeat where one of the overlapping reads starts upstream of the interspersed repeat and the second read extends downstream from the interspersed repeat. In the case of a tandem repeat region composed of identical copies of the repeated motif, a contiguous assembly requires a read to fully span the entire block of tandem repeats because the correct registration between two reads that are anchored on opposite sides of the block of tandem repeats cannot be determined. Specifically, bridging a tandem repeat block with two reads from opposite sides, rather than fully spanning the region with a single read, can lead to an expansion or a collapse of the number of repeated units in the tandem repeat region (as shown in FIG. 4).

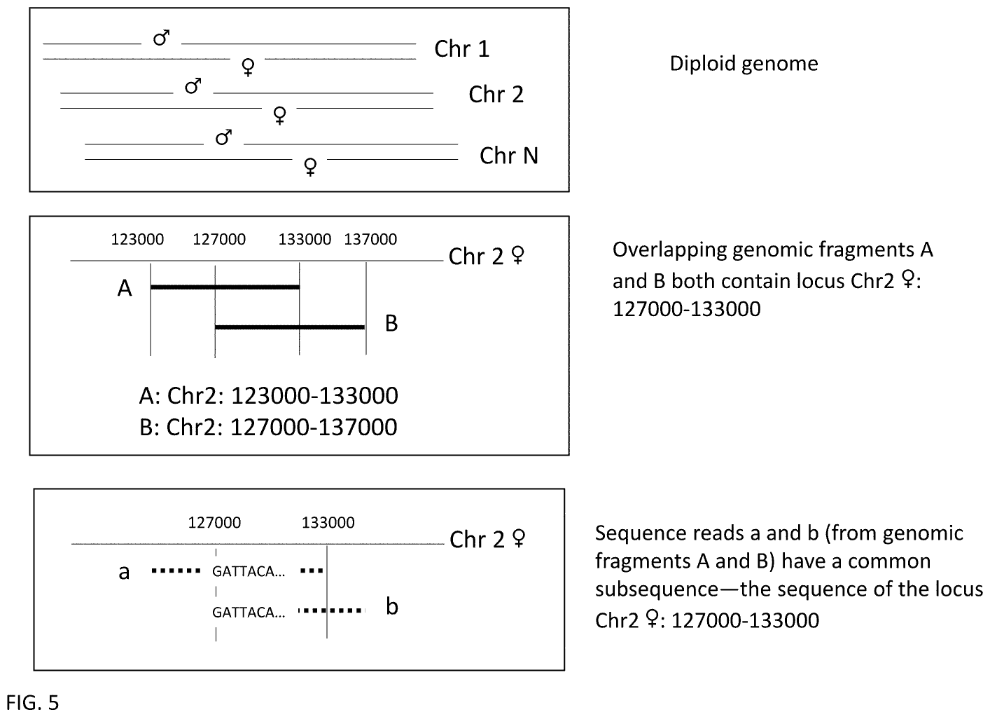

In addition to the problem of repetitive elements occurring at distinct loci, there is also the problem of homozygosity in polyploid genomes, which contain multiple homologous copies of each chromosome. This is represented in the top panel of FIG. 5, with the paternal chromosome indicated by ♂ and the maternal chromosome indicated by ♀. The human genome is an example of a highly homozygous diploid genome, with differences between homologous chromosomes of less than 0.1%. The desired assembly of a polyploid genome is a set of contigs, where each contig represents a complete chromosome and each homologous chromosome represented by a distinct contig. As shown in the middle panel of FIG. 5, genomic fragments A and B include the common locus 127000-133300 derived the maternal chromosome 2. Their respective sequence reads a and b thus include the common subsequence of this shared maternal genomic locus, i.e., the sequence of the locus 127000-133000. The overlap of these sequence reads (shown in the bottom panel) accurately reflects the underlying genomic structure.

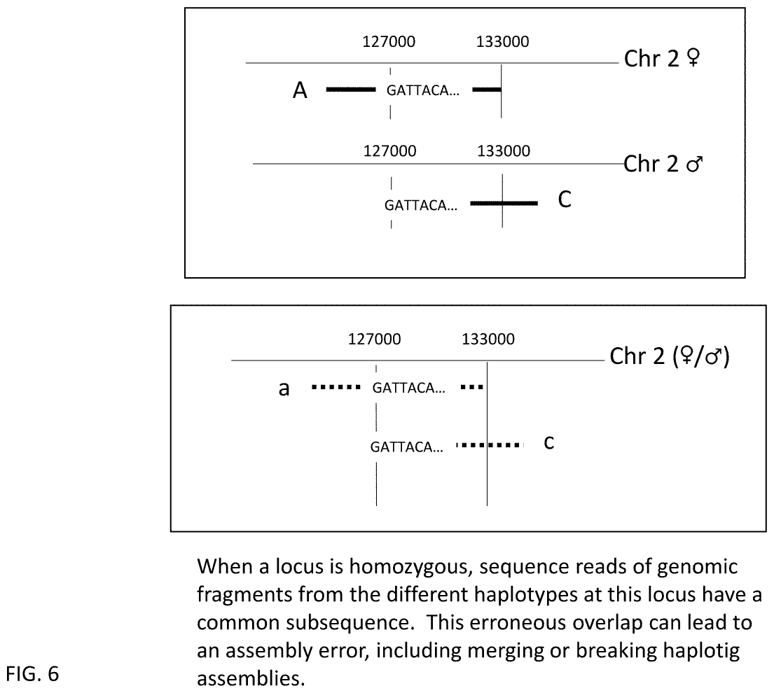

At a given locus, two alleles, i.e., homologous loci on two distinct homologous chromosomes, are said to be homozygous if they have identical sequence at that locus. As shown in FIG. 6, genomic fragments A and C include a homozygous locus in chromosome 2: nucleotides 127000-133000 of the maternal chromosome 2 and nucleotides 127000-133000 of the paternal chromosome. Their respective sequence reads a and c thus include the common subsequence of this homozygous genomic locus, i.e., the sequence of the locus 127000-133000 of the maternal and paternal chromosomes. The overlap of these sequence reads (shown in the bottom panel) does not accurately reflect the underlying genomic structure. This erroneous overlap can result in an assembly error that merges the maternal and paternal contigs at this locus or that beaks them. Correct diploid assembly requires a determination of not only whether two reads are derived from the same genomic locus, but also whether they are derived from the same haplotype of that locus. The assumption that reads sharing a common sequence are derived from genomic fragments arising from the same allele may be rendered invalid by homozygous regions. Assembly is limited by the length of repetitive elements and homozygous loci relative to read lengths when the common sequences are identical. Long reads are required to span long regions where two haplotypes are identical, extending into regions where there is enough variation between the haplotypes to easily distinguish them.

The ability to distinguish highly similar, non-identical sequences is a function not only of the length of these sequences, but also the extent of the similarity. Noisy reads may need to be very long indeed to fully span a region of moderate similarity that extends over a long distance in the genome. Highly accurate reads of only moderate length, however, may also assemble the same region by spanning numerous shorter regions of identical sequence if the accuracy is sufficient to distinguish intervening regions of only moderate similarity, thus anchoring the two ends of the read.

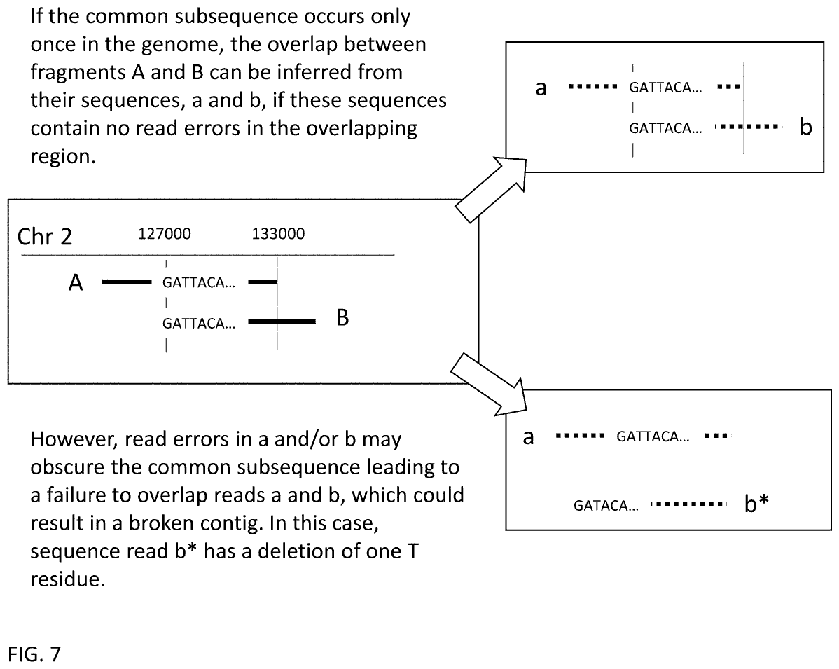

Reads arising from two distinct but highly similar sequences can be distinguished when the accuracy of two reads is so high that the number of differences between the reads is significantly higher than the expected number of read errors. However, it is also possible to identify that two reads came from genomic fragments with distinct nucleotide sequences with even higher similarity by examining the types of difference between the two reads. For example, the read errors in many long-read platforms are predominantly indels. For example, in FIG. 7, when error-free reads a and b for genomic fragments A and B are analyzed, they are correctly overlapped (top right panel), whereas when error-free sequence read a and error-containing sequence read b* are analyzed, the homopolymer deletion error in b* (i.e., the removal of “T” from the “TT” homopolymer) results in a failure to overlap reads that should be overlapped (based on the fact that they are derived from overlapping genomic fragments A and B in chromosome 2). In contrast to this predominant form of sequence read errors, the true (or biological) differences between two highly similar genomic loci or heterozygous alleles is most often single-nucleotide substitutions. Therefore, if the only differences between a pair of reads are homopolymer indels, one could infer that the reads came from genomic fragments with identical sequences and that the differences are sequence read errors. Conversely, if the only differences between a pair of reads were single-nucleotide substitutions, one could infer that the reads came from genomic fragments that are highly similar but not overlapping, e.g., genomic fragments from different alleles. In the extreme case, we can segregate two reads derived from genomic fragments that differ at only one position into their distinct haplotypes (i.e., two reads that differ by one single nucleotide variation, or SNV).

Noise Filtering: True Biological Variation Vs. Sequencing Read Errors

An important aspect of noise filtering is recognizing and exploiting the situation when the signal and noise lie in essentially orthogonal directions in some coordinate space. With respect to genome assembly processes, the signal we are considering is the true biological variation between repetitive sequence elements or haplotypes (e.g., SNVs) and the noise is sequencing read errors (e.g., homopolymer indels).

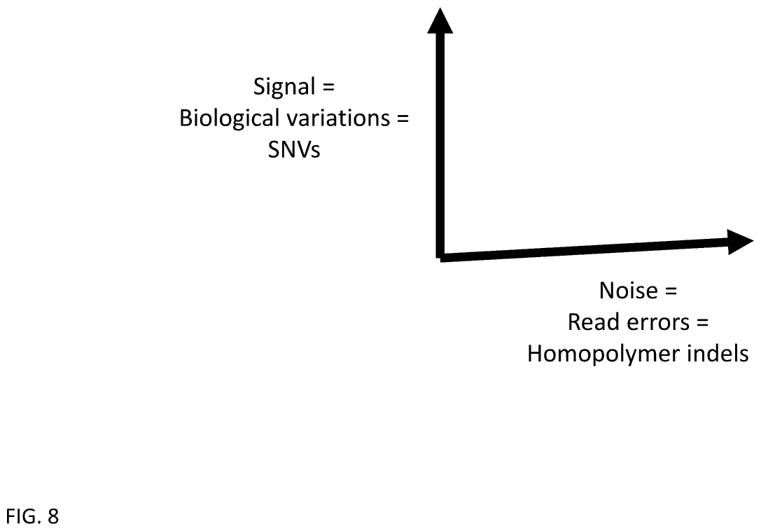

The relationship between these signal and noise vectors is shown in FIG. 8. In this figure, two approximately orthogonal vectors representing signal and noise are displayed, where the signal vector represents biological differences that can be used to identify when two genomic fragments are not overlapping, and thus belong to different genomic loci and/or haplotypes (in this case SNVs), and the noise vector represents sequence read errors that prevent identification of two genomic fragments that overlap, and thus belong to the same genomic loci and/or haplotype (in this case homopolymer indels). In genomes, the majority of biological differences between highly similar sequences belonging to different haplotypes and/or different genomic loci are single nucleotide variants (SNVs). In many sequencing platforms, read errors are predominantly homopolymer indels (see Table 1 in Wenger, A., et al., Jan. 13, 2019 “Highly-accurate long-read sequencing improves variant detection and assembly of a human genome” BioRxiv, doi.org/10.1101/519025; hereby incorporated herein by reference in its entirety for all purposes). In contrast, nucleotide substitution errors, which could be mistaken for biological SNVs, are relatively rare. The difference between biological variation and read errors presents an opportunity for filtering. The approximate orthogonality, depicted in this figure, between signal and noise, means that noise can be suppressed without significantly diminishing the strength of the signal.

The assembly process consists of finding pairs of reads (R1, R2) that form long dovetail alignments, where suffix of R1 aligns to a prefix of R2 or vice versa. An alignment whose length exceeds a defined threshold and some sequence similarity is assumed to be a true overlap and is used in the assembly. When the reads are error-free (i.e., no noise), the alignments of suffix and prefix are exact string matches. Gusfield, et al. (Gusfield, Dan, Gad M. Landau, and Baruch Schieber. “An efficient algorithm for the all pairs suffix-prefix problem.” Information Processing Letters 41.4 (1992): 181-185; hereby incorporated herein by reference in its entirety) describes an algorithm using suffix trees that solves the all pairs suffix-prefix problem with a time complexity that is linear in the sum of the inputs (i.e., the sum of the read lengths) and the sum of the outputs (i.e., the square of the number of reads). Because detecting pairwise overlaps between reads is recognized as the rate-limiting step in genome assembly, a method that accelerates this step leads to significantly faster assembly.

We use the well-characterized differences between haplotypes in a typical human genome as a model of biological variation (the “signal”). A human genome is comprised of roughly 3 billion positions, where the homologous sequence on the paternal and maternal chromosome are aligned. For this rough analysis of rates of variation, we ignore differences in the sex chromosomes (X and Y) in a male. In a typical person, there are roughly 3 million single-nucleotide variations (SNVs; substitutions of one nucleotide for another) and roughly 300 thousand insertion and deletion variations (indels). The corresponding rates for SNV and indels are 1 per 1000 and 1 per 10,000 (see Chaisson, Mark J P, et al. “Multi-platform discovery of haplotype-resolved structural variation in human genomes.” bioRxiv (2018): 193144; hereby incorporated herein by reference in its entirety).

When assembling a genome or genomic region from a collection of sequencing reads, it is important to overlap two reads when the prefix of one read and the suffix of a second read are derived from the same genomic fragment (a “dovetail” overlap). To prevent spurious dovetail overlaps of reads where the identical sequence occurs in two different genomic fragments (i.e., sequences from different locations in the genome are identical to one another), the overlap length can be set to exceed the length of all (or a majority of) such identical genomic fragments, e.g., from about 1,000 to about 7,000 nucleotides. It is noted that adjustment of the overlap length parameter can be done by a user to address specific issues related to what is known about the genome being sequenced and/or the sequencing platform being used, and as such, no strict threshold for the overlap length is intended. In general, increasing the minimum overlap length parameter increases the specificity of overlap detection while reducing sensitivity. Assemblies formed with higher sensitivity, i.e., at a lower minimum overlap length, have higher contiguity but may lead to joining two reads derived from non-overlapping genomic fragments. In addition, even when the overlapping of sequence reads is determined correctly (i.e., it is not the result of a sequence read error), two reads from different haplotypes that themselves do not overlap may nonetheless both be joined to a third read that overlaps with a homozygous region shared by both haplotypes. For example, two reads having homozygous suffix regions can both overlap with the same third read whose prefix includes all or part of this homozygous region. In this case, two different haplotypes may be undesirably merged into a single connected component. Fortunately, these merges can often be resolved in subsequent steps of the assembly process, e.g., by pruning the connected component of the third read to break this haplotype merging.

In the genome assembly methods described herein, we wish to avoid overlapping two sequencing reads that do not share an identical contiguous subsequence of sufficient length. Any difference between sequencing reads indicate that the two reads are derived from polynucleotide substrates that contained different, non-overlapping genomic fragments, either different regions of the genome or from different haplotypes of the same region. In either case, incorrectly overlapping such sequencing reads would introduce an error in the assembly, e.g., a diploid assembly.

In some instances, it is possible that two independent genomic fragments, i.e., genomic fragments that occur in distinct locations in the genome or that are distinct haplotypes at the same locus, are identical over a length that exceeds the length threshold for scoring overlapping sequencing reads. When such genomic fragments occur at distinct genomic positions, the false overlap of the sequencing reads derived from those genomic fragments introduces an assembly error. When such genomic fragments occur in distinct haplotypes at the same genomic position, the false overlap of the sequencing reads derived from those haplotypes causes the two haplotypes to merge, resulting in the end of a contiguous phase block in the assembly (a phase block is a region in a genome assembly where the haplotype sequences are separable, e.g., the maternal and paternal sequences are resolved). The relative phase of two distinct phase blocks interrupted by a homozygous block cannot be determined. False overlaps induced by identical sequence cannot be avoided in the absence of additional information at a scale longer than the provided read length.

Our current goal is to detect with high sensitivity and specificity the smallest possible sequence difference between two genomic fragments, i.e., a single substitution or indel, within two sequence reads, e.g., two SMCS reads.

Filtering out noise (i.e., sequencing read errors) allows successful detection of the underlying biological variation and prevents the types of assembly and consensus errors described above. The resulting assemblies are more accurate, more contiguous, and have improved haplotype resolution, both in the length of contiguous phase blocks and in consensus accuracy.

In many sequencing platforms, homopolymer indels present a distinct challenge. Consider a genome sequence containing five consecutive A's (i.e., AAAAA). If the positions of the five A's cannot be distinguished in the read, then there are five ways to generate the read sequence AAAA, i.e., by deleting any one of the five A's. Similarly, there are six ways to generate the read sequence AAAAAA, i.e., by inserting an A before the first A, after the last A, or between any two As. Because the degeneracy of indels increases linearly with the length of the homopolymer, the single-pass error rate also increases with homopolymer length.

The consensus sequence (e.g., SMCS read) for a homopolymer is particularly error-prone because of the high single-pass error rate in these regions as compared to non-homopolymer indel errors (e.g., substitutions). Thus, the distribution of errors in consensus reads are significantly skewed toward homopolymer indels and away from other types of errors. The enrichment of homopolymer indel errors as the predominant type of error in consensus sequence reads increases both with the length of the homopolymer region and the number of reads used to generate the consensus. For SMCS reads, the higher the number of subreads, the higher the predominance of homopolymer indel errors as a fraction of total sequence errors. For example, in SMCS reads formed by 10 subreads by Pacific Biosciences' SEQUEL® nucleic acid sequencing instrument, roughly 99% of the errors are homopolymer indels.

The prevalence of homopolymer indel errors means that high read coverage (a combination of single- and multi-molecule reads) is required to reliably determine the lengths of long homopolymers. However, the concentration of SMCS read errors into a single channel (i.e., homopolymer indels) provides an opportunity for noise filtering during the genome assembly process.

Recall that haplotype variants in a human genome are 90% SNVs and 10% indels. Roughly one-fourth of these occur in homopolymers. Thus, only a few percent of true human haplotype variation (signal) is homopolymer indels. Therefore, when we observe two aligned reads, e.g., SMCS reads, that differ only by indels in homopolymer regions, it is likely that the differences are read errors (noise) and that the reads are derived from the same genomic fragment.

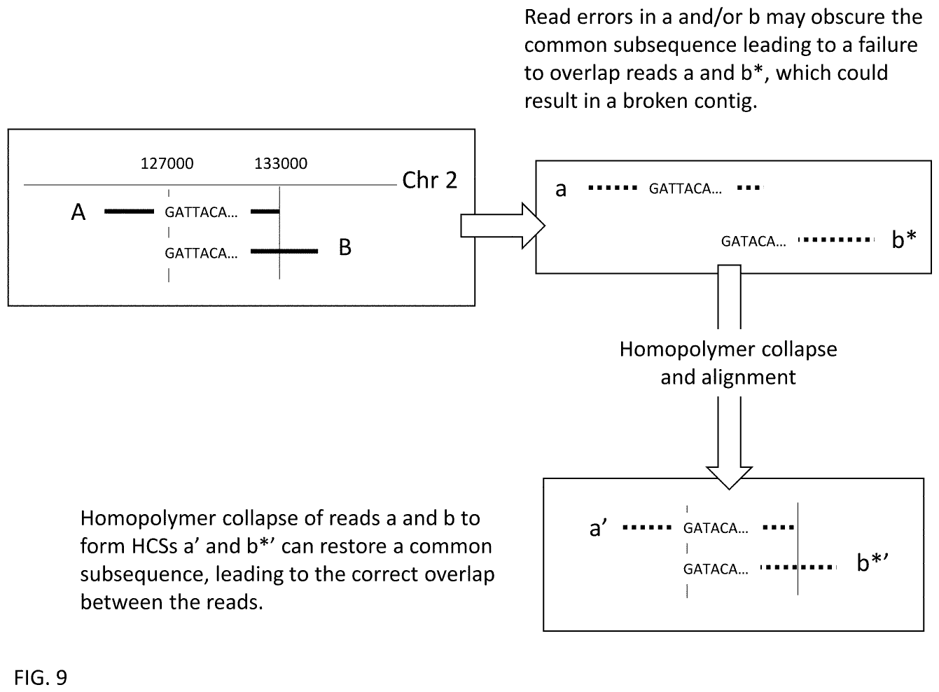

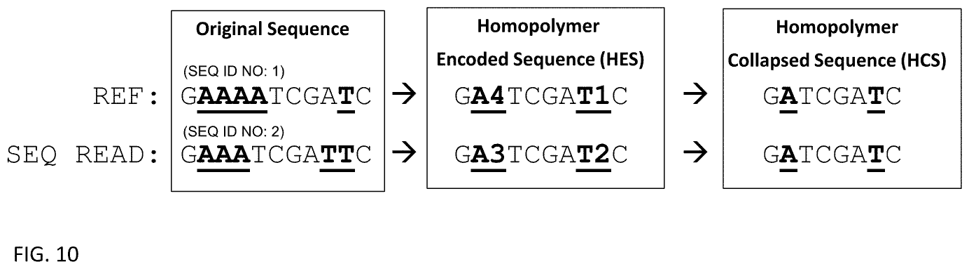

This property provides the basis of a method for suppressing read errors (noise) to reveal subtle biological sequence variation (signal). Specifically, the sequence alignment methods described herein eliminate the confounding effect of homopolymer indel errors by reducing homopolymer strings in sequence reads to a single base of the same type (termed homopolymer collapse) prior to aligning. Reads that differ only by homopolymer indels become identical after homopolymer collapse and can be paired by exact string matching. For example, in FIG. 9, reads a and b* shown in the top right panel (same as in FIG. 7) can be correctly overlapped by first converting them to their homopolymer-collapsed forms, which masks the homopolymer indel error in b* (see bottom right panel in FIG. 8). Because of the highly skewed error profile (predominantly homopolymer indels), sequence reads that align by exact string matching after homopolymer collapse over a significant portion of their length (e.g., 100, 200, 300, 400, 500, 750, 1000, 2000, 3000, 4000, 5000 bases or more) are assumed to be derived from the same genomic fragment and overlapped. The combination of many such exact sequence overlaps forms the basis of a draft assembly.

In current assembly processes for polyploid genomes, a draft assembly undergoes “polishing” to resolve discordances in the multiple sequence alignment of aligned reads to produce a consensus sequence for each haplotype. In many cases, polishing a polyploid genome assembly involves an iterative process of partitioning reads into haplotypes and then calling a consensus sequence for each partition.

In contrast to this iterative polishing process, the draft assembly that results from exact string matches of overlapping homopolymer-collapsed reads as described herein is largely already haplotype-resolved, with the exception that long homozygous regions that are not spanned by a single sequence read can cause haplotypes to merge. In the exact string matching-based methods described herein, distinct haplotype blocks are formed by removing sequence reads that fall completely within regions of overlap in which all of the aligned positions agree (i.e., for each position in a sequence read, if there is only one base represented in all of the overlapping reads at those positions, the read is removed). Once these reads are removed, at every position in a haplotype block, all reads belonging to that haplotype have the same nucleotide at that position. Thus, for a diploid genome, there should be at most two haplotype blocks for a genomic region. At this point, consensus is trivial for each haplotype block because, by its very construction, the homopolymer-collapsed reads mapped to the same haplotype agree at every aligned position. Therefore, the homopolymer-collapsed consensus sequence for each haplotype is determined by simply reading off the unanimous basecall at each aligned position. Thus, in regions of the genome where multiple haplotype blocks are formed, this process results in consensus sequences for each distinct haplotypes, which by definition differ at one or more positions. For the homozygous regions of the genome that interrupt haplotype blocks, because no single sequence read spanned the entire homozygous region, reads from both haplotypes produce a single unanimous consensus.

After breaking the genome into heterozygous and homozygous regions and assigning a consensus homopolymer-collapsed sequence to each homozygous region and to each haplotype in a phase block, all that remains to generate a full polyploid assembly is to re-expand the homopolymer-collapsed sequences by making consensus calls for the haplotype-resolved homopolymer lengths. As noted herein, when each sequence read is collapsed, the lengths of its homopolymers are recorded. For each homopolymer, the set of lengths for that homopolymer in the aligned reads for a given haplotype is used to determine a consensus length call (examples for this process are described below).

As indicated elsewhere herein, aspects of the present disclosure employ single-molecule consensus sequence (SMCS) reads, which are formed by obtaining multiple individual reads derived from a single original polynucleotide fragment (e.g., a single genomic fragment) and combining them to form a single consensus sequence for that original polynucleotide fragment. Like multi-molecule consensus, in which reads from different original polynucleotide fragments are aligned and analyzed, the redundancy in the multiple reads that are used to generate a SMCS provides a mechanism for suppressing read noise (i.e., sequencing errors). Unlike multi-molecule consensus, the multiple reads used to form a SMCS read are known to arise from the same original polynucleotide fragment, so the possibility of mapping errors is eliminated. This allows the SMCS read to be “polished” to high accuracy before they are overlapped with other SMCS reads. The high accuracy of SMCS reads may be sufficient to distinguish sequences derived from distinct but highly similar genomic fragments from each other that cannot be distinguished by lower accuracy single-pass reads.

Errors in SMCS reads are a direct consequence of the errors in the single-pass reads from which they are derived. In a platform where indels are the dominant error type (in single-pass reads), indels will also be the dominant error type in SMCS reads. Error types that occur less frequently in single-pass reads (e.g., substitutions) tend to “wash out” rapidly from the SMCS read. In general, each type of single-pass error washes out exponentially from the SMCS read with increasing number of subreads. The exponential factor determining the rate of a particular error type in a SMCS read is the rate of that error type in single-pass reads. Thus, variations in the rates of various types of single-pass read errors are amplified when comparing error rates in SMCS reads.

### Computer Implemented Analysis

Aspects of the methods presented herein may be embodied in whole or in part as software recorded on fixed media for use in a computer (or computer system). The computer may be any electronic device having at least one processor (e.g., CPU and the like), a memory, input/output (I/O), and a data repository. The CPU, the memory, the I/O and the data repository may be connected via a system bus or buses, or alternatively using any type of communication connection. The computer may also include a network interface for wired and/or wireless communication. In one embodiment, computer may comprise a personal computer (e.g., desktop, laptop, tablet etc.), a server, a client computer, or wearable device. In another embodiment the computer may comprise any type of information appliance for interacting with a remote data application and could include such devices as an internet-enabled television, cell phone, and the like.

The processor controls operation of the computer and may read information (e.g., instructions and/or data) from the memory and/or a data repository and execute the instructions accordingly to implement the exemplary embodiments. The term processor is intended to include one processor, multiple processors, or one or more processors with multiple cores.

The I/O may include any type of input devices such as a keyboard, a mouse, a microphone, etc., and any type of output devices such as a monitor and a printer, for example. In an embodiment where the computer comprises a server, the output devices may be coupled to a local client computer.

In general, the subject disclosure provides computer-implemented methods that employ homopolymer-collapsed sequences (HCSs) to improve aligning sequences, determining consensus sequences, mapping sequences to a reference, and/or sequence assembly processes, e.g., in de novo assembly of genomes. As defined above, HCSs are sequences derived from a parent sequence in which each instance of multiple consecutive identical nucleotides in the parent sequence is replaced by a single nucleotide of the same type. For example, the HCS of the polynucleotide sequence AATGGGCCG is ATGCG. It is noted that the length of each collapsed homopolymer is stored for each HCS, so this information is not lost. These stored homopolymer lengths are used in downstream analyses, e.g., to make haplotype-resolved consensus homopolymer length calls for polishing a draft genome assembly.

As described herein, homopolymer collapse allows for greatly improved sequence analysis when applied to sequencing platforms for which the predominant type of sequencing error is homopolymer indel errors. As defined above, homopolymer indel errors are those that insert or delete a nucleotide that is identical to an adjacent, and correct, nucleotide in a sequencing read. Applying homopolymer collapse to a sequencing read containing homopolymer indel errors and to a reference sequence to which it is being compared (or the polynucleotide substrate sequence from which it is derived) results in a perfect match between the sequences. In other words, the homopolymer indel errors are masked and thus do not negatively impact sequence alignment algorithms. In addition, homopolymer collapse of multiple sequencing reads allows computer-implemented assembly of contigs and genomes that use exact string matching, rather than error-tolerant algorithms that rely on a similarity threshold or exact matching of short k-mer seeds (e.g., k<30) and chaining.

The homopolymer collapse/exact string-matching method detailed herein is distinguished from k-mer matching approaches as follows. In current practice, k-mer matching is used to identify short common subsequences shared by two reads which may be part of an overlapping region between two reads. However, the two reads may be judged to overlap (i.e., to be derived from overlapping genomic fragments) even though the aligned region contains sequence differences between the two reads, i.e., differences in sequence that are between the perfect k-mer matched regions identified. Thus, k-mer matching is error-tolerant. In contrast, exact string-matching is not error-tolerant, and thus is not merely k-mer matching as currently practiced with a longer value of k. Instead, exact string-matching judges two reads to overlap only if the overlapping region between the two reads is identical, i.e., there are no differences between the reads in the entirety of the overlapping region. Because exact string-matching is not error-tolerant, overlap determination by exact-string matching has a higher specificity than k-mer matching. In addition, because it is not error-tolerant, exact string matching of homopolymer-collapsed sequences results in significantly faster alignment, consensus, and assembly processes (described below). Moreover, for SMCS reads and other read types where homopolymer indels are the predominant error type (e.g., nanopore sequencing), exact string-matching has higher sensitivity and specificity for identifying true overlaps between the genomic sequences from which a pair of reads is derived.

In some embodiments of the present disclosure, the sequence reads employed are single molecule consensus sequences (SMCS) reads, which can be derived from any sequencing platform in which generating SMCS reads is possible, e.g., SMRT® Sequencing and nanopore sequencing platforms. In general, SMCS reads are consensus sequences generated by analyzing multiple single-pass sequence reads derived from the same original polynucleotide substrate molecule, e.g., by repeated sequencing of the original polynucleotide substrate (as in SMRT® Sequencing) or by sequencing multiple copies of the original polynucleotide substrate (as in sequencing linear concatemers generated by rolling circle amplification, or other means, using nanopore sequencing). (See, e.g., FIG. 1 and its description above.) It is noted that concatemers can be sequenced in SMRT® Sequencing applications by generating SMRTBELL® polynucleotide substrates that each include concatemers derived from a single polynucleotide substrate and/or by generating multiple SMRTBELL® polynucleotide substrates each of which include a copy from the same original polynucleotide substrate. Moreover, sequencing topologically circular polynucleotide substrates can be done using certain nanopore sequencing methodologies, e.g., from Genia, now part of Roche (see Fuller et al., 2016, PNAS 113(19):5233-8, hereby incorporated herein by reference in its entirety). Limitations in this regard are thus not intended.

It is noted here that while SMCS reads are described for use in the subject methods, the methods described herein are not limited to SMCS reads. Indeed, the methods described herein are applicable to any sequence reads for which homopolymer indel errors are a significant or predominant sequence read error type, and thus a confounding issue for genome assembly, including single-pass sequence reads. No limitation in this regard is intended.

Current algorithms for mapping and alignment of reads involve a fast screening step based upon detecting one or more perfect k-mer matches between sequences followed by a dynamic programming step to find the optimal sequence alignment. The fast screening step involves a trade-off between specificity and sensitivity which is modulated by the choice of k, the length of the k-mer. Larger values of k make it less likely that two sequences would overlap by random chance. Smaller values of k make it less likely that sequencing read errors would obscure a match to the correct target (i.e., the locus from which the read was derived or another read derived from the same locus). Reducing the number of differences between a sequencing read and its target (e.g., other sequencing reads, reference sequence, etc.) means that larger values of k can be used without losing sensitivity to correct matches. However, as noted above, current k-mer alignment algorithms are error-tolerant and thus require some form polishing to arrive at consensus for overlapping regions of sequence reads that can include sequence differences outside of the aligned k-mer regions.

Dynamic programming is a method for exploring all alignments between two sequences in a time that scales with the product of the sequence lengths. If the sequences are error-free, the alignment can be found in time that scales with the length of the longer sequence (i.e., linear time). By categorizing HCSs of sequence reads as error free, e.g., HCSs of SMCS reads, we can exploit this feature of dynamic programming by requiring exact string-matching for aligning sequences (as opposed to using current k-mer matching).

Signal and noise in sequencing data are not perfectly “orthogonal”. For example, while the vast majority of read errors (noise) in a sequencing platform are homopolymer indels, it is also the case that there are occasionally genomic fragments that have a biological homopolymer indel difference (signal), e.g., a genomic fragment from a first haplotype at a genomic locus will differ from a genomic fragment from the second haplotype at the same genomic locus by the length of a homopolymer sequence. Based on our current understanding of the human genome, the sequences of two 5 kb genomic fragments with 99.9% similarity will, on average, differ by roughly five nucleotide substitutions and roughly 0.5 indels. With respect to the indels, about 0.4 of the 0.5 indels occur outside homopolymers and about 0.1 of the 0.5 indels occur within homopolymers. A false 5 kb overlap between two SMCS reads occurs when the 5 kb overlap region has no substitution differences, no indels outside homopolymers, and one or more homopolymer indels. Thus, false overlaps of 5 kb between SMCS reads are highly unlikely, even when the genomic fragments from which the SMCS reads were derived are highly similar. The vast majority of false overlaps result in failure to recognize heterozygous variation, resulting in collapse of phase blocks, which occurs most often outside of coding regions. False overlaps that lead to incorrect assembly of the genome may occur within repetitive regions where large numbers of repeat elements share very high sequence similarity, such as centromeres, but otherwise are very unlikely to occur. Even so, the ability to detect a single-base difference between genomic fragments (most often a substitution) substantially improves the mean length of phase blocks in highly homozygous genomes, such as the human genome.

In certain embodiments, the present disclosure leverages the unique properties of long SMCS reads (e.g., 10-15 kb or longer) that can be generated from long read sequencing technologies, e.g., those that produce polymerase reads of 50 kb, 75, kb, 100 kb, 150 kb or longer. Specifically, the long read-lengths result in the ability to obtain a high number of subreads from original polynucleotide substrates of ˜10-15 kb in length (e.g., 4, 5, 6, 7, 8, 9, or 10 subreads or more) which can be used to generate SMCS reads having 99 to 99.99% accuracy or greater. In some embodiments, the polynucleotide substrates analyzed according to the present disclosure are derived from genomic DNA samples, where in some cases the genomic DNA sample is from a polyploid organism, e.g., a plant, fungal, animal, or human genome. In other cases, the sample is a metagenomic sample containing multiple different microorganisms, e.g., bacterial, protozoan, yeast, or other single-celled organisms. These SMCS reads greatly reduce non-homopolymer indel errors, including substitution errors (errors that change one base to a different base, e.g., reading polynucleotide substrate sequence AGCTG as AGATG) and indel errors that insert or delete a nucleotide base that is different from the two adjacent bases (e.g., reading polynucleotide substrate AGCTG as either ATGCTG or ACTG). For SMRT® Sequencing, we have found that errors of all types are reduced exponentially with the number of passes.

Based on the discussion above, it is clear that most errors in SMCS reads (e.g., generated from ˜4-10 subreads or more) are homopolymer indels. Because most biological variants are single nucleotide variants (substitutions of one base for another), SMCS read error types show very low overlap with true biological variants. Therefore, removal of homopolymer indels in SMCS reads by homopolymer collapse (thereby generating HCS reads) preferentially removes sequencing platform-based errors while leaving true biological variants. Filtering out these errors will thus improve numerous downstream sequence analysis algorithms, from mapping and alignment to de novo genome assembly. Once any desired downstream alignment of HCS reads is complete, the collapsed homopolymers of each HCS read can be expanded (based on their length in the original SMCS read). The expanded homopolymer regions of the SMCS reads can then be analyzed to determine a consensus length at each different position. These consensus homopolymer lengths can then be added back to any consensus sequence generated from the process using the HCS reads (e.g., assembly, alignment, and/or any resulting consensus sequence).

The Figures and their descriptions below are meant to exemplify certain embodiments of the methods disclosed herein and are not meant to be limiting. For example, while the description below is related to HCSs from SMCS reads, HCSs from single-pass sequence reads in which homopolymer indel errors are a predominant or significant error type can be employed.

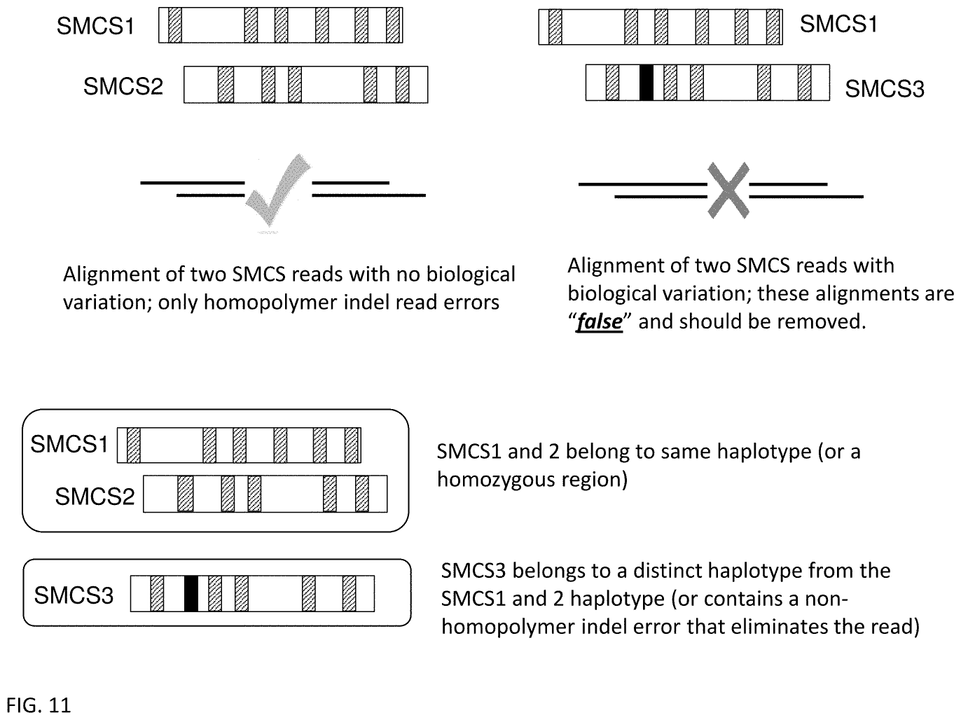

FIG. 11 shows an example of aligning pairs of SMCS reads after filtering out homopolymer indels, which represent the vast majority of sequencing errors. Shaded blocks represent homopolymer indel errors, the predominant error type in SMCSs. The solid block in SMCS3 represents a single nucleotide variation (SNV) that identifies SMCS3 as being derived from a different haplotype than SMCS1 and SMCS2. Homopolymer indel errors are masked by homopolymer collapse and ignored when determining whether two reads are derived from the same haplotype. Because the only differences between SMCS1 and SMCS2 are homopolymer indels and homopolymer indels are assumed to be read errors during the overlap step of assembly, SMCS1 and SMCS2 are assumed to be derived from the same haplotype (the same genomic fragment). In contrast, the single nucleotide substitution difference is assumed to be a true biological difference between the haplotypes.

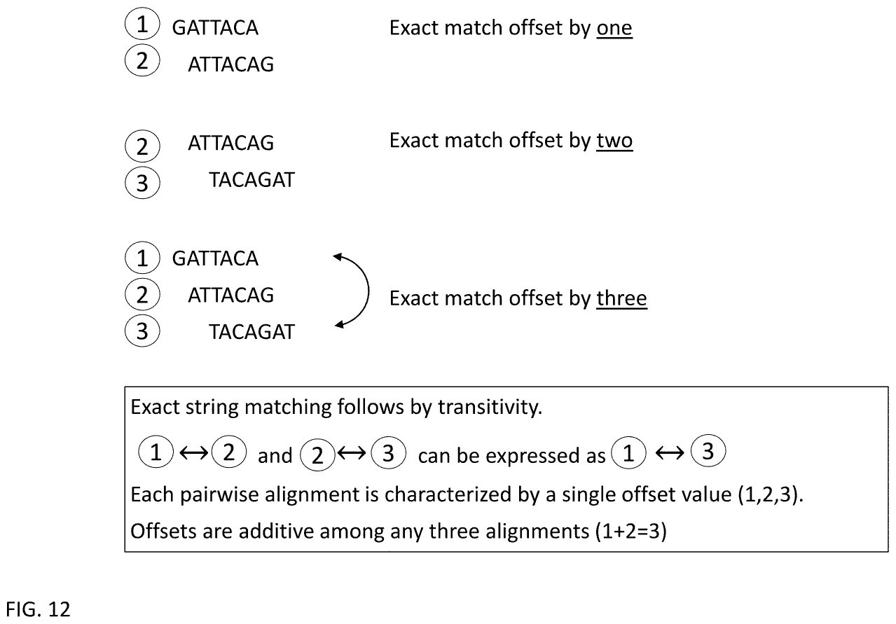

FIG. 12 shows a toy example of a multiple sequence alignment formed from pairwise exact string matches of SMCS reads. Pairwise exact string matches can be characterized simply by an integer offset. Multiple sequence alignments, which in general are quite complicated, are trivial for exact string matched reads from the same haplotype. Exact string matching is transitive, and offsets are additive.

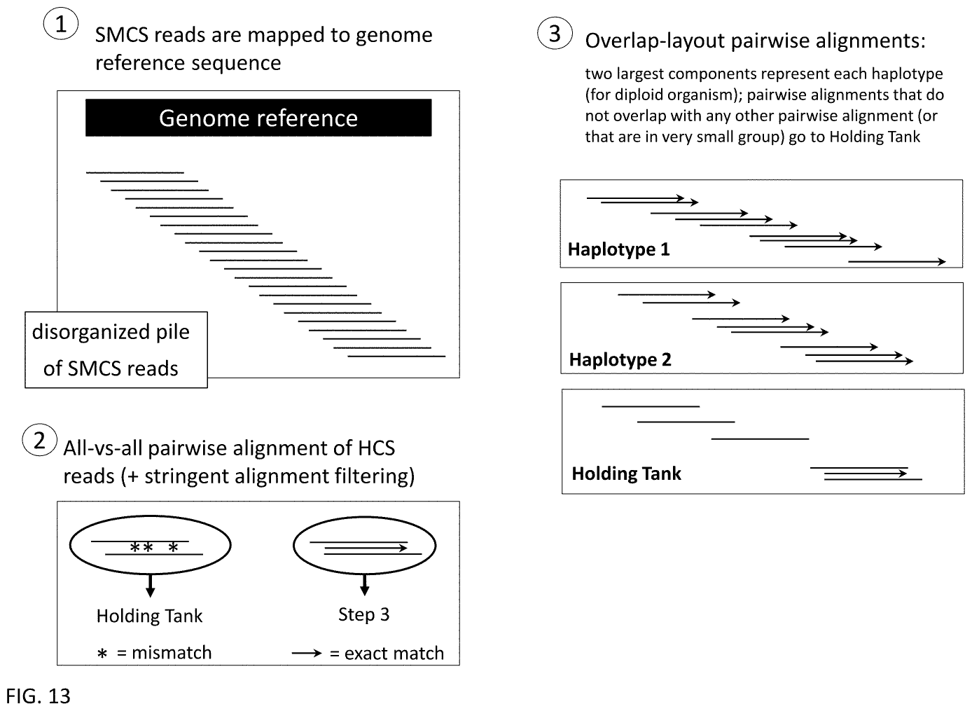

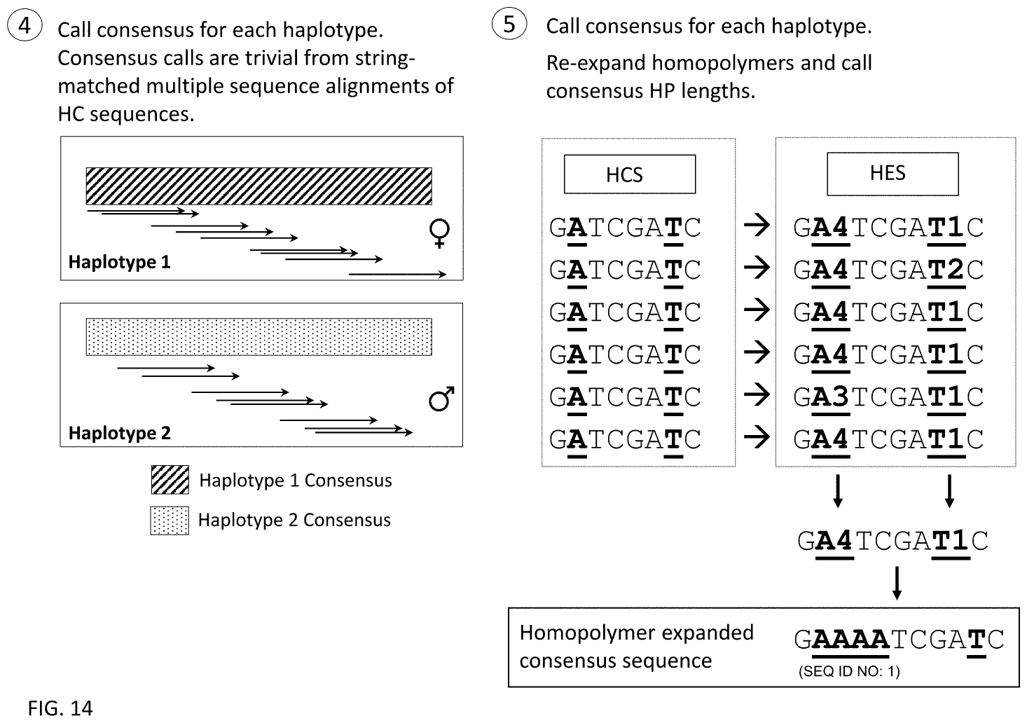

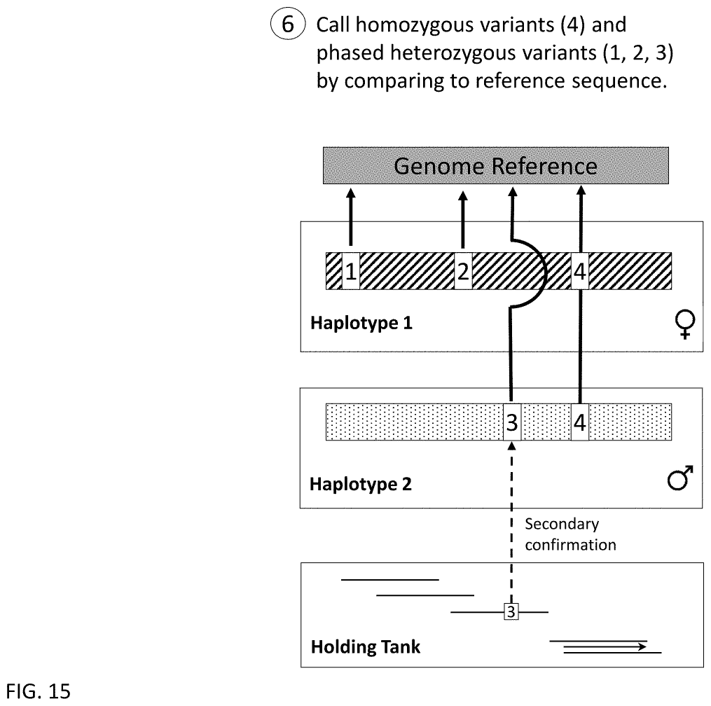

FIGS. 13 to 15 show one embodiment of a sequence analysis pipeline that employs homopolymer collapse and exact alignment mapping to segregate SMCS reads into haplotypes. While these figures depict haplotype segregation of a diploid genome (e.g., a human genome), this analysis pipeline is suitable for any sequence analysis for which segregating SMCS reads into groups of sequences derived from the same original genome/polynucleotide substrate is desired, e.g., in metagenomic sequence analysis. The analysis pipeline also addresses genomes with higher ploidy such as tetraploid (n=4), hexaploid (n=6), or octaploid (n=8). No limitation in this regard is intended.

In step 1 of the pipeline in FIG. 13, SMCS reads are selected that map to a specific region(s) of a reference genome. This step is not a necessary feature of the algorithm, but was employed here to construct a problem of limited size, i.e., the haplotype-resolved assembly of the highly similar SMN1 and SMN2 loci, allowing for an easily understood demonstration of the algorithm's utility. This initial mapping can be performed with relatively low stringency to maximize the number of SMCS reads used for downstream analysis, as reads that are incorrectly mapped to the region are easily filtered out during the assembly process. The region or regions can be selected by a user, e.g., a region associated, or predicted to be associated, with a phenotype (e.g., a disease phenotype). Once a subset of SMCS reads that map to a region of interest (or to multiple regions of interest) have been selected/obtained (denoted in FIG. 13 as a “disorganized pile of SMCS reads”), they are converted to HCS reads and subjected to an “all-vs-all” pairwise alignment with stringent filtering, as described herein (step 2 in FIG. 13). For example, the alignments can be filtered such that the alignment region is (1) at least ¼ to ½ the length of the average sequence read length (or of a threshold minimal length that is predicted to span homozygous regions in the genome under study, e.g., ˜1 kb to ˜5 kb), and (2) an exact match between the suffix of one read and the prefix of another read. The alignment on the right of step 2 meets these criteria and is processed in step 3, with the aligned region depicted with a right-facing arrow. All pairwise alignments that do not meet these criteria are discarded or placed in a holding tank. SMCS reads that contain any read errors other than homopolymer indels will not form exact string matches with other reads and will also be placed in the holding tank. The alignment on the left of step 2 is placed in a holding tank because it has multiple mismatches in the aligned region (denoted by “*”). The aligned regions (denoted by arrows) of all of the pairwise alignments that meet this filtering requirement are compared and segregated using an overlap-layout algorithm in step 3, where pairwise alignments that have an exact overlap in their respective alignment regions are segregated to the same group (or haplotype, as in FIG. 13; haplotypes 1 and 2). The reads belonging to a distinct haplotype are determined by treating the reads and alignments between the reads as the vertices and edges in a graph, respectively, and finding the connected components of this graph. In this case, each alignment between a pair of reads indicates that two reads may belong to the same haplotype, but also provides the relative offset between the start positions of the reads that would be required to line up the corresponding positions where the sequences match. These pairwise offsets can be used to lay out a set of connected reads along a common coordinate axis as shown in step 3. In this case, each panel contains a set of reads that belong to the same haplotype. As a result, at any given position in the multiple sequence alignment, all reads that cover that position have the same basecall at that position. Regions that form pairwise alignments that do not overlap with any other regions that form pairwise alignments are placed into the holding tank. These orphan pairwise alignment regions could be from SMCS reads that were mistakenly mapped to the region of interest in step 1 and/or could be from SMCS reads of polynucleotide contaminants or sample preparation artifacts (e.g., from inadvertent mixing of initial genomic DNA samples or the generation of chimeric polynucleotide substrates and/or amplification artifacts during sample preparation, etc.). The criteria for placing pairwise alignments (and/or their SMCS reads) into the holding tank can be determined by a user and may be based on what is known about the genomic sample, e.g., ploidy or expected number of organisms in a metagenomic sample, sample preparation details, etc. In this way, one can group reads by haplotype from observed differences in pairwise alignments.

As shown in FIG. 14, a consensus sequence is then generated for each haplotype, or group of overlapping sequences (step 4). The consensus sequence for the haplotype is determined by reading off the basecall at each position in sequence. The consensus sequences here represent the homopolymer-collapsed consensus sequence for each haplotype/group. After generating the consensus sequences from the HCSs, the homopolymer-collapsed regions can be expanded to generate homopolymer-expanded consensus sequences in step 5. This process involves attaching the homopolymer length that was observed and recorded at each collapsed position in each read, transforming a set of aligned homopolymer-collapsed reads (HCS) into a set of aligned homopolymer-expanded reads (HES). Notice that the alignment of these reads is retained because we “expand” each homopolymer not by representing the homopolymer by a string of repeated nucleotides but rather as a basecall and a repeat number. For example, a homopolymer of 4 A's is represented by “A4” rather than “AAAA” (top HES read in step 5). The right panel of FIG. 14 shows two positions in the multiple sequence alignments where the (expanded) homopolymer lengths in the reads are not unanimous. In this example, to form homopolymer length calls at these positions we find the floor of the median value. We take the floor of the median because the consensus homopolymer length must be integer-valued. We select the floor rather the ceiling because shorter homopolymers occur more often the longer ones. By calling the homopolymer length at each position in the homopolymer-collapsed consensus sequence, we form a run-length encoded representation of a homopolymer-expanded consensus sequence. Now, we expand each run-length encoded homopolymer as a string of repeated nucleotides, e.g., transforming “A4” to “AAAA”, to produce the final homopolymer-expanded consensus sequence, as depicted in FIG. 14.

One example of homopolymer expansion includes the following. First, a vector of homopolymer lengths is associated with each position in the homopolymer-collapsed sequence, where (i) the number of elements in the vector is the number of trimmed HCSs covering that position in the multiple sequence alignment, and (ii) each component of the vector is the observed length of the homopolymer in the original read at that position in the HCS. For example, in FIG. 14, the vector for the “A” nucleotide at position 2 in the HCS is derived from the corresponding position in the HES, and thus is: 4, 4, 4, 4, 3, 4. Next, the consensus homopolymer length for each position in the homopolymer-collapsed sequence is calculated as the floor of the median of the components of the vector of homopolymer lengths associated with that position, e.g., the floor of the median of the lengths derived from the corresponding positions in the HESs. In FIG. 14, this value is 4, since the floor of the median value of the series 3, 4, 4, 4, 4, 4 is 4. Finally, each position in the homopolymer-collapsed sequence is replaced with a homopolymer string N of the same nucleotide, where N is the consensus homopolymer length calculated for that position.

As shown in FIG. 15, once the homopolymer-expanded consensus sequences are called in step 5, these consensus sequences are then compared to the genome reference sequence in step 6 (e.g., the genomic domain used to select the initial SMCS reads) to call any heterozygous variants (denoted 1, 2, and 3) and/or homozygous variants (denoted 4). In some embodiments, the reads in the holding tank can be used to confirm the calling of variants if there are regions of low coverage in the consensus sequence. This is shown in FIG. 15 as a dotted arrow from variant 3 in a HCS read in the holding tank that supports the calling of variant 3 in haplotype 2 consensus. It is noted that the variant positions may occur in homopolymer regions, since they have been expanded. Analyzing reads in the holding tank by expanding their homopolymer regions might also aid in determining consensus homopolymer lengths should this be advantageous.

Perfected reads, e.g., SMCS reads whose errors are fully masked by homopolymer collapse (as defined above), participate in diploid assembly through exact string matches to other perfected reads. Two perfected reads are overlapped in the assembly process when the prefix of the polynucleotide substrate HCS from which one reads was derived is the suffix of the polynucleotide substrate HCS from which the other read was derived, forming a perfect dovetail alignment. Such alignments are what is desired in generating an accurate genome assembly.

Therefore, to maintain accuracy in a genome assembly, we want to exclude reads that have not been perfected from participating in the assembly process. The requirement that an overlap is formed between two SMCS only when their HCSs match exactly has the effect of excluding many reads that carry errors that were not masked out by homopolymer collapse. Except for rare coincidences, a read that contains (unmasked) errors near both termini will not match exactly to any other read.

However, we must also consider the case of a read that has a single (unmasked) error. Roughly speaking, such a read has a perfected half that will overlap with other perfected reads, but the other half carries an error and will not overlap with other reads. This read is kept in the analysis because it forms a perfect dovetail assembly with perfected read. One possible outcome is that such a read will terminate a contig in the assembly, since only one side of the read forms a perfect dovetail alignment. Another possibility that such a read will cause a “spur”, which resembles a distinct haplotype variant that forms a branch that is separated from other perfected reads.

To avoid the detrimental effects of including a non-perfected SMCS reads of this type in an alignment process, we remove these errors from such SMCS reads prior to the layout step of assembly by trimming off any positions at the end of a read that fail to overlap with any other read. This quality control step makes certain that all bases used for the assembly process are represented by at least two separate SMCS reads at that position. In embodiments where the threshold overlap length is at least half of the average read length, positions that are not covered by at least one overlap are likely to lie at the ends of reads.

Before the layout step (e.g., step 3 in FIG. 13), we first generate a graph representing the pairwise overlaps between reads. Each read is represented by a vertex in the graph. Each overlap between a pair of reads is represented by an edge between the corresponding vertices. Under ideal circumstances, a connected component of the graph would represent one chromosome (e.g., one haplotype of a genome). In a diploid genome, there would be one component for each paternal chromosome and one component for each maternal chromosome. Distinct chromosomes would be represented by distinct connected components.

In many cases, however, a chromosome is represented by multiple connected components as a result of fragmentation in the assembly. Fragmentation can be caused by systematic and/or random coverage dropouts, leaving some positions that are not covered by any reads. In the presently disclosed algorithm, contiguity of the assembly at a position requires that the position is covered by at least two perfected SMCS reads.

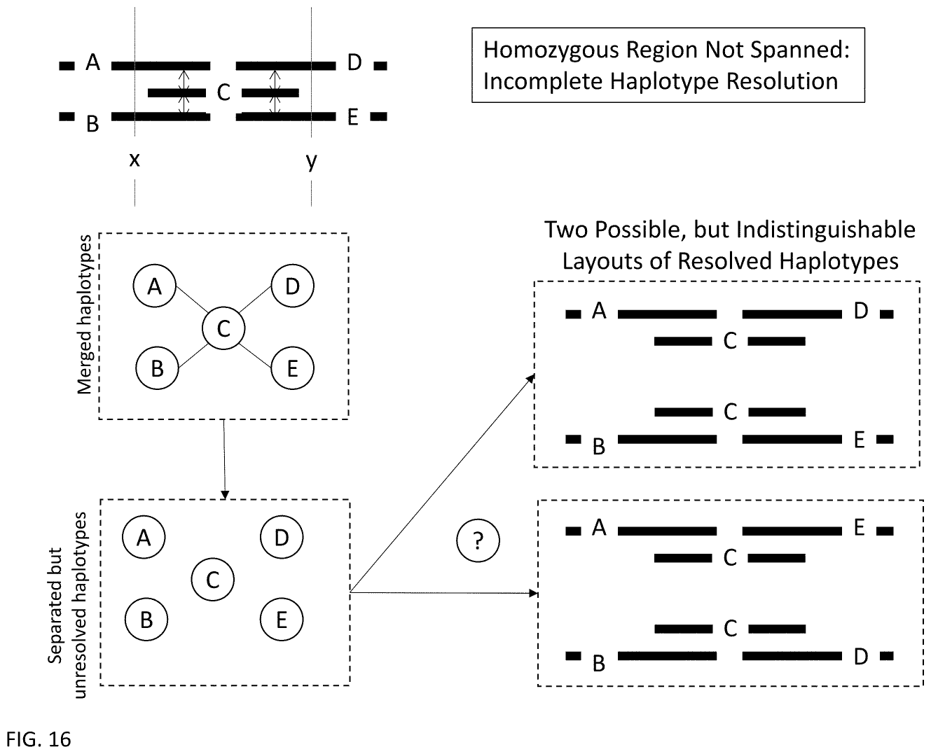

In addition to fragmentation, connected components may represent the merging of pieces from multiple chromosomes. Most frequently, a merged connected component is caused by a homozygous region that is shared by two or more haplotypes. For example, as shown in FIG. 16, read A and read B belong to different haplotypes, contain one or more positions where the haplotypes vary (denoted by the “x” position), and thus do not overlap. However, both read A and read B overlap with a third read C. The overlap between A and C contains only homozygous positions, i.e., where the two haplotypes have the same sequence. Likewise, the overlap between B and C contains only homozygous positions. In this case, reads A and B that belong to distinct haplotypes are merged into the same connected component through their mutual overlap to read C in a homozygous region of the genome. In FIG. 16, reads D and E, which vary at position “y”, overlap with the other end of read C in a similar manner. Thus, read C contains only homozygous positions at this locus in the genome; it contains neither x nor y.

This alignment scenario results in the graph labeled “Merged haplotypes” in FIG. 16. Such merged haplotypes (or “connected components”) are separated by inducing a subgraph of the connected component by removing edges representing an overlap that contain only homozygous positions (e.g., by removing node C from the graph). This process is referred to as pruning. For example, the overlaps between A and C and B and C would be removed as would the overlaps between D and C and E and C. If there are no SMCS reads that contain both position x and y, then removing read C would break the graph into four connected components, as shown in the “Separated but unresolved haplotypes” box. For a diploid genome, there are two possible layouts of the pair of homologous haplotypes: 1) A connected to D via C and B connected to E via C (shown in the top right layout of FIG. 16); or 2) A connected to E via C and B connected to D via C (shown in the bottom right layout of FIG. 16). The homozygous region between flanking resolved haplotype regions thus induces a haplotig break, but not a contig break (i.e., haplotypes cannot be resolved, but the contig through this region is still intact).

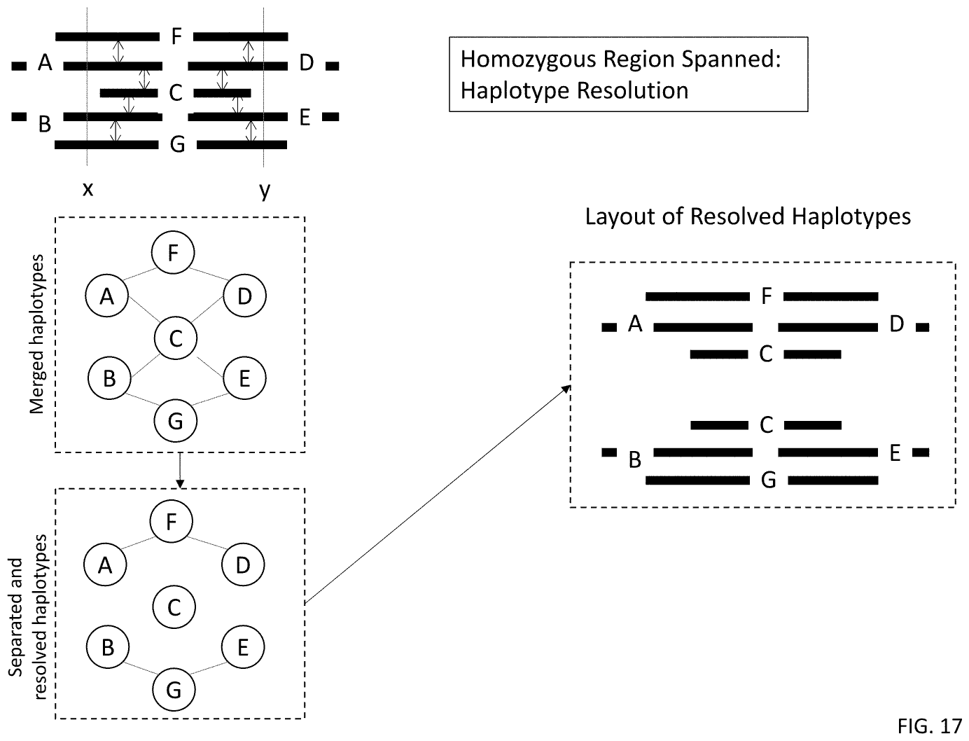

FIG. 17 shows a situation that is related to the one depicted in FIG. 16, except that the collection of sequence reads (shown top left) includes reads F and G, each of which span both positions x and y, i.e. spanning the homozygous region. These reads can be used to resolve the two haplotypes. If F overlaps with reads A and D (meaning that it includes the same variant at positions x and y as read A and D) and read G overlaps with reads B and E (meaning that it includes the same variant at positions x and y as read B and E), then removing the edges connected to the vertex associated with read C (i.e., pruning) induces a graph with two connected components, one for each contiguous haplotype (shown at the right).

### Example: SMN1/SMN2 Genomic Regions

In the following example, the survival of motor neuron 1 and 2 loci (SMN1 and SMN2) are analyzed according to one embodiment of the present disclosure. SMN1 and SMN2 are part of a 500 kb inverted duplication on chromosome 5q13, with SMN1 being the telomeric copy and SMN2 being the centromeric copy. These genes encode the same protein, SMN. This duplicated region contains at least four genes and repetitive elements which make it prone to rearrangements and deletions. The repetitiveness and complexity of the sequence have also caused difficulty in determining the organization of this genomic region. Mutations in SMN1, the telomeric copy, are associated with spinal muscular atrophy (also known as Werdnig-Hoffmann Disease or Kugelberg-Welander Disease); mutations in the centromeric copy, SMN2, do not lead to disease. The centromeric copy may be a modifier of disease caused by mutation in the telomeric copy. Mutations in both SMN1 and SMN2 result in embryonic death. The critical sequence difference between the two genes is a single nucleotide in exon 7, which is thought to be an exon splice enhancer. The nine exons of both the telomeric and centromeric copies are designated historically as exon 1, 2a, 2b, and 3-8. It is thought that gene conversion events may involve the two genes, leading to varying copy numbers of each gene. The protein encoded by this gene localizes to both the cytoplasm and the nucleus. Within the nucleus, the protein localizes to subnuclear bodies called gems which are found near coiled bodies containing high concentrations of small ribonucleoproteins (snRNPs). This protein forms heteromeric complexes with proteins such as SIP1 and GEMIN4, and also interacts with several proteins known to be involved in the biogenesis of snRNPs, such as hnRNP U protein and the small nucleolar RNA binding protein. Two transcript variants encoding distinct isoforms have been described.

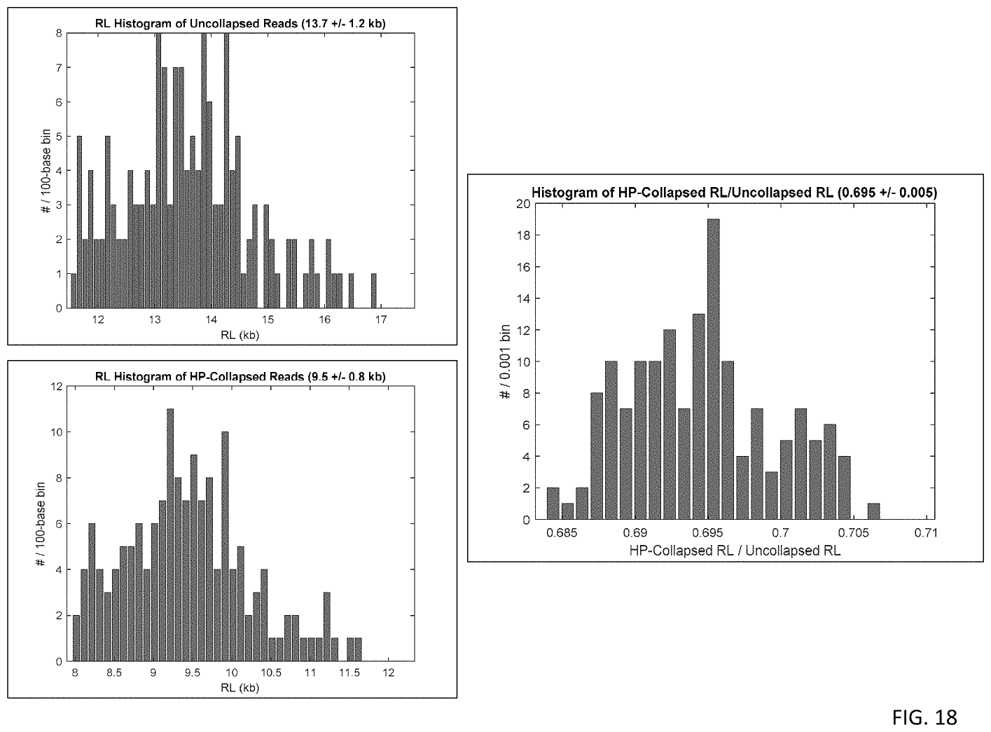

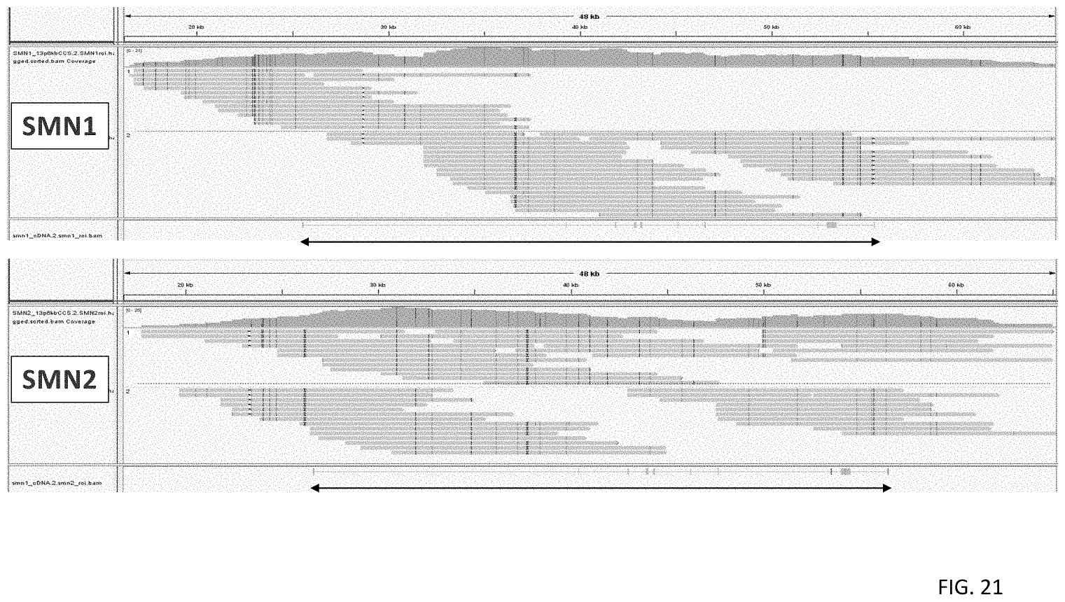

FIGS. 18 to 20 show preliminary results in the diploid assembly of SMN1 and SMN2 regions from a set of SMCS reads. FIG. 21 shows the final result. The data and the assembly process are described in further detail below.

We first obtained human genomic (HG002) DNA SMCS reads from fragments in a narrow band (+/−1 kb) centered around 13.5 kb (these reads are described in Wenger, A., et al., Jan. 13, 2019 “Highly-accurate long-read sequencing improves variant detection and assembly of a human genome” BioRxiv, doi.org/10.1101/519025; hereby incorporated herein by reference in its entirety for all purposes). We used a subset of these reads that mapped to either SMN1 or SMN2 with minimap2 under relatively low stringency (some may map to both, given their very high sequence similarity). A histogram of the SMN-mapped SMCS read lengths selected for this analysis is shown in the top left panel of FIG. 18. This resulted in 154 SMCS reads being selected.

Next, we made a reverse-complement copy of each SMCS read, forming a set of 308 SMCS reads. Note that the initial collection of SMCS reads represents reads of genomic fragments from both strands the genome. We consider two genomic fragments to be “overlapping” if one fragment overlaps with the reverse-complement of another fragment. By making two “mirror-image” copies of each read, we will form two mirror-image assemblies from the collection of reads. The genomic reference (arbitrarily) represents one of the two strands, and so we retain the assembly that corresponds to the reference strand. Then, we generated a homopolymer-collapsed sequence (HCS) for each SMCS read. A histogram of the HCS lengths is shown in the bottom left panel of FIG. 18. The mean HCS length is 9.5 kb. A histogram of the ratios between HCS and SMCS lengths is shown in the right panel of FIG. 18. Homopolymer-collapse reduced the majority of the SMCS reads to 69-70% of their original length. For comparison, collapse of strings generated by drawing four letters with equal probability independently at random would reduce the strings to 75% of their original length. Next, we performed an all-vs-all pairwise alignment of these 308 SMCS reads. A total of 494 alignments were formed between pairs of reads. A pair of reads has an alignment between them if the suffix of one read is identical to the prefix of another read and the length of this common subsequence is longer than the minimum overlap length. Here, we chose a minimum overlap length of 6 kb, a value which just exceeds half the longest HCS in the collection. These candidate alignments were then segregated into groups based on their connectivity. For this step, aligned reads were represented as a graph in which the read is a vertex and the alignment is a directed edge. The directed edge points from the read whose suffix matches another read's prefix.

The graph induced by the 494 alignments between the 308 reads had twelve connected components between 200 reads—six pairs of components where the members of a pair were mirror-images of each other. The other 108 reads were singletons—reads that did not overlap with any other read. Most likely, these singleton reads failed to overlap with other reads because they were corrupted by one or more read errors. Because we chose a minimum overlap greater than half the length of any HCS, a single read error at the midpoint of the read would cause the read to fail to overlap with any other read—that is, except for the highly unlikely possibility that another read were identical at 6000 positions or more, contained no errors in any of these positions, except for one of exactly the same type at exactly the same position. More often, read errors at both ends of the read exclude it from an assembly process constructed from overlapping reads based upon exact string matches.

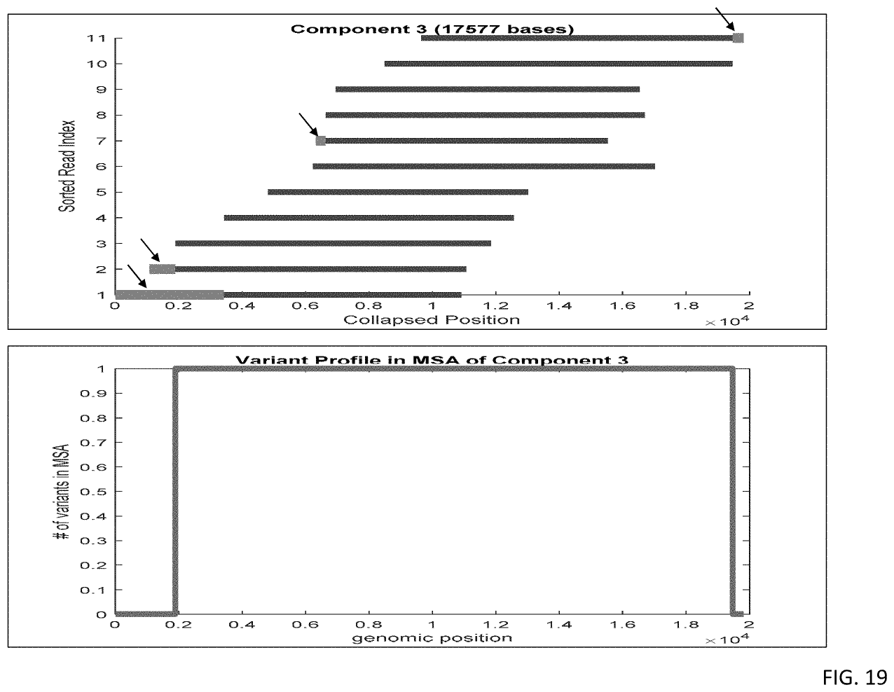

The process of determining the connected components of the graph also generates a layout of the reads within each component. Components are formed by making a breadth-first traversal from an arbitrary read and assigning this read an arbitrary coordinate value of zero. The prefix of each read newly reached in the traversal matches the suffix of a read already reached, so that the coordinate of each new read is at least as large as the reads that already belong to a traversal. When a traversal from a new read touches a read that has already been assigned to a component, the two components are merged. The coordinates of all reads in the newly touched component are increased by a fixed offset so that the coordinates in the merged component are self-consistent. The top panel of FIG. 19 shows the layout for component 3 comprised of 11 HCSs derived from SMCS reads. This layout covers approximately 20 kb, but only 17,577 bases after trimming. Slightly thicker lines extending from the ends of four HCSs (arrows) show regions of the reads that were trimmed because these regions did not overlap with any other HCS in the collection, most likely because of a read error. These trimmed bases do not contribute to the assembly. The locations at the left and right ends in the layout that are not represented by at least 2 HCS reads (only covered by one of the HCS reads) are trimmed and not used to form the consensus. The bottom panel of FIG. 19 shows the number of variant base calls in the multiple sequence alignment induced by the layout of the HCSs. In this case, at every aligned position that is covered by at least two reads (i.e., excluding trimmed regions), every read covering that position has the same basecall. The reads provide unanimous consensus for each basecall in the consensus sequence. Another way to describe this multiple sequence alignment is that each constituent HCS is a proper (exact) substring of the homopolymer-collapsed consensus sequence. The values of zero in the variant profile in the bottom panel of FIG. 19 corresponds to positions that are covered only by trimmed regions of reads.

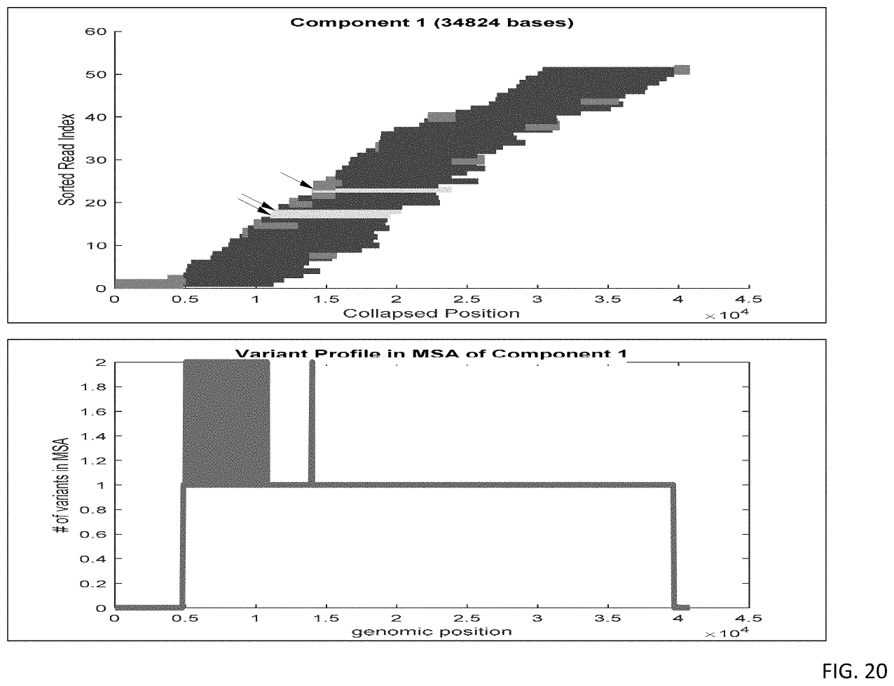

In contrast to the situation shown in FIG. 19, FIG. 20 shows a connected component that contains two merged haplotypes. In FIG. 20, 54 HCSs form a connected component that spans nearly 40 kb before trimming. The variant profile for this component, shown in the bottom panel of FIG. 20, shows that while most positions are concordant between all reads at that position, some of the positions contain reads that disagree. At each of the discordant positions, the reads can be divided into two groups defined by the basecall they contain at that position. The two groups represent two distinct haplotypes. Three reads (colored light grey in the top panel of FIG. 20, arrows) are responsible for merging these two haplotypes. Each of these three reads overlaps with a pair of reads that belong to different haplotypes. This occurs because the overlapping region of each read has a sequence that is common to both haplotypes. After identifying all such reads that merge haplotypes, we remove them from the graph, recalculate the connected components and generate two new connected components that are haplotype-resolved.

FIG. 21 shows the final diploid assembly in which the consensus sequences representing each connected component are mapped to the sequences for SMN1 and SMN2 that appear in the human genome reference GrCh38. Before the assembly process, most individual SMCS reads couldn't be confidently mapped to either SMN1 or SMN2 because the similarity between the reference sequences is higher than similarity between the SMCS read and either reference sequence. Despite the high accuracy of the SMCS reads, it was ambiguous whether the genomic fragment from which the SMCS read was derived was itself derived from the SMN1 locus or the SMN2 locus. Even homopolymer collapse of the SMCS reads, which eliminates most of the read errors, did not resolve this ambiguity for most reads. The mapping shown in FIG. 21 is possible only because a few nucleotides in Exons 7 and 8 distinguish SMN1 from SMN2. This allowed us to map a limited number of reads to the proper locus, but only in this region. However, because we have a diploid assembly, the connection of these “mappable” reads to other reads that belong to the same haplotype anchors the entire haplotig to the proper locus. The marked variant positions between SMCS reads and the reference allow us to make variant calls across the entire length of both loci. The concordance of these variants across multiple aligned reads provides strong evidence of the correctness of these variant calls. In many positions, heterozygous variation is clearly apparent where two haplotypes can be clearly identified.

The SMN1 and SMN2 loci are notoriously difficult to assemble because of their high similarity and high homozygosity. In a recent high-quality assembly derived from these reads, current assemblers were unable to map reads to any exons from either SMN1 or SMN2. [This is noted in FIG. 2c of Wenger et al., which lists the SMN1 and SMN2 exons as being 0% mappable (Wenger, A., et al., Jan. 13, 2019 “Highly-accurate long-read sequencing improves variant detection and assembly of a human genome” BioRxiv, doi.org/10.1101/519025; hereby incorporated herein by reference in its entirety for all purposes).]

It will be readily apparent to one of ordinary skill in the relevant arts that other suitable modifications and adaptations to the methods and compositions described herein can be made without departing from the scope of the invention or any embodiment thereof. Having now described the present invention in detail, the same will be more clearly understood by reference to the following Examples, which are included herewith for purposes of illustration only and are not intended to be limiting of the invention.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1. A method for assembling a genome or a chromosome, the method comprising:
obtaining a plurality of sequence reads for genomic fragments for the genome or the chromosome from a genomic sample, wherein each sequence read comprises a sequence of basecalls;
generating a homopolymer-collapsed sequence (HCS) and a corresponding homopolymer encoded sequence (HES) for each sequence read in the plurality of sequence reads, thereby generating a plurality of HCS reads and a plurality of HES reads;
generating a plurality of suffix/prefix exact string matches of the plurality of HCS reads, wherein a length of the exact string match is at or above a minimum length;
generating an overlap graph from the plurality of HCS reads, wherein the overlap graph comprises a plurality of vertices connected by a plurality of directed edges, and wherein each vertex of the graph represents an HCS read in the plurality of HCS reads and each directed edge in the plurality of directed edges is from a first vertex to a second vertex in the plurality of vertices whenever (i) there is a suffice/prefix exact string match between the first and second HCS reads represented by the first vertex and the second vertex, and (ii) the suffix of the first vertex exactly matches the prefix of the second vertex;
identifying one or more connected components in the overlap graph;
generating a multiple sequence alignment for each connected component in the one or more connected components, thereby generating one or more multiple sequence alignments;
generating a homopolymer-collapsed consensus sequence by concatenating a basecall at each aligned position in a first multiple sequence alignment in the one or more multiple sequence alignments;
associating a vector of homopolymer lengths for each position in the homopolymer-collapsed consensus sequence, wherein:
(i) the number of elements in the vector is the number of HCS reads covering that position in the multiple sequence alignment, and
(ii) each component of the vector is the length of the homopolymer in the corresponding HES at that position;
assigning a consensus homopolymer length for each position in the homopolymer-collapsed consensus sequence; and
replacing each position in the homopolymer-collapsed consensus sequence with a homopolymer string formed by N successive copies of nucleotide at that position, wherein N is the assigned consensus homopolymer length calculated for that position, to generate a homopolymer-expanded consensus sequence, thereby assembling the genome or chromosome.

2. (canceled)

3. The method of claim 1, wherein the minimum length is from 0.5 kb to 10 kb.

4. The method of claim 3, wherein the minimum length is from 5 kb to 8 kb.

5. The method of claim 4, wherein the minimum length is from 6 kb to 7 kb.

6. The method of claim 1, wherein the minimum length is at least half the length of the average length of the plurality of HCS reads.

7. The method of claim 1, wherein the plurality of sequence reads are generated in a single molecule sequencing-by-synthesis reaction.

8. The method of claim 7, wherein the single molecule sequencing by synthesis reaction is a Single Molecule, Real-Time (SMRT) Sequencing reaction.

9. The method of claim 1, wherein the plurality of sequence reads are generated in a single molecule nanopore sequencing reaction.

10. The method of claim 1, wherein the plurality of sequence reads is a plurality of single molecule consensus sequences (SMCSs).

11. The method of claim 10, wherein the SMCSs are generated from at least 4 subreads.

12. The method of claim 11, wherein the at least 4 subreads are generated in a single molecule sequencing reaction from a concatemeric polynucleotide substrate.

13. The method of claim 12, wherein the at least 4 subreads are generated in a single molecule sequencing-by-synthesis reaction.

14. The method of claim 12, wherein the at least 4 subreads are generated in a single molecule nanopore-based sequencing reaction.

15. The method of claim 11, wherein the at least 4 subreads are generated in a single molecule sequencing-by-synthesis reaction from a circular or topologically circular polynucleotide substrate.

16. The method of claim 1, wherein the genome is a human genome.

17. The method of claim 1, wherein the genomic sample comprises multiple different genomes, the method further comprising generating assemblies for multiple of the different genomes.

18. The method of claim 17, wherein the genomic sample is a metagenomic sample comprising multiple microbial genomes.

19. The method of claim 1, wherein HCSs that are not placed into the one or more connected components are placed into a holding bin that is used to verify variant calls in the genome or chromosome.

20. The method of claim 1, wherein the plurality of sequence reads are pre-selected to map to one or more genomic regions of interest prior to generating the plurality of HCSs.

21. The method of claim 20, wherein the pre-selection mapping is done with a low-stringency sequence similarity search.

22. The method of claim 20, wherein the one or more genomic regions of interest comprises a first and a second genomic loci having high sequence similarity to one another.

23. The method of claim 22, the method further comprising generating separate consensus sequences for the first and second genomic loci.

24. The method of claim 20, wherein the one or more genomic regions of interest comprises a genomic locus having a highly repetitive region.

25. The method of claim 1, wherein the method is a method for de novo genome assembly.

26. The method of claim 25, wherein the de novo genome assembly is a fully or partially haplotype resolved assembly of a polyploid genome.

27. A system for assembling a genome or a chromosome, comprising:
a memory;
input/output; and
a processor coupled to the memory, wherein the system is configured to:
receive a plurality of sequence reads for genomic fragments for the genome or the chromosome from a genomic sample, wherein each sequence read comprises a sequence of basecalls;
generate a homopolymer-collapsed sequence (HCS) and a corresponding homopolymer encoded sequence (HES) for each sequence read in the plurality of sequence reads, thereby generating a plurality of HCS reads and a plurality of HES reads;
generate a plurality of suffix/prefix exact string matches of the plurality of HCS reads, wherein a length of the exact string match is at or above a minimum length;
generate an overlap graph from the plurality of HCS reads wherein the overlap graph comprises a plurality of vertices connected by a plurality of directed edges, and wherein each vertex of the graph represents an HCS read in the plurality of HCS reads and each directed edge in the plurality of directed edges is from a first vertex to a second vertex in the plurality of vertices whenever (i) there is a suffice/prefix exact string match between the first and second HCS reads represented by the first vertex and the second vertex and (ii) the suffix of the first vertex exactly matches the prefix of the second vertex;
identify one or more connected components in the overlap graph;
generate a multiple sequence alignment for each connected component in the one or more connected components, thereby generating one or more multiple sequence alignments;
generate a homopolymer-collapsed consensus sequence by concatenating a basecall at each aligned position in a first multiple sequence alignment in the one or more multiple sequence alignments;
associate a vector of homopolymer lengths for each position in the homopolymer-collapsed consensus sequence, wherein:
(i) the number of elements in the vector is the number of HCS reads covering that position in the first multiple sequence alignment, and
(ii) each component of the vector is the length of the homopolymer in the corresponding HES at that position;
assign a consensus homopolymer length for each position in the homopolymer-collapsed consensus sequence;
replace each position in the homopolymer-collapsed consensus sequence with a homopolymer string formed by N successive copies of nucleotide at that position, wherein N is the assigned consensus homopolymer length calculated for that position, to generate a homopolymer-expanded consensus sequence; and
provide the homopolymer-expanded consensus sequence to a user, thereby assembling the genome or chromosome.

28. (canceled)
